(19) United States
(12) Patent Application Publication
(10) Pub. No.: US 2023/0123341 A1
(43) Pub. Date: Apr. 20, 2023

(54) METHODS AND SYSTEMS FOR SCORING FOODS AND RECIPES

(71) Applicants: Vivek Singh, Oakville (CA); Archana Singh, Oakville (CA)

(72) Inventors: Vivek Singh, Oakville (CA); Archana Singh, Oakville (CA)

(21) Appl. No.: 18/046,933

(22) Filed: Oct. 16, 2022

Related U.S. Application Data
(60) Provisional application No. 63/256,535, filed on Oct. 16, 2021.

(51) Int. Cl. G16H 20/60

(52) U.S. Cl. CPC G16H 20/60

(57) ABSTRACT

Computer-implemented methods, systems, and products for scoring the suitability of a recipe or food to an end-user having one or more medical conditions are provided. The methods and systems can include: the nutrient content of the food or a food created following the steps of the recipe; the chronic or health or medical conditions or general health that the target user may be interested in; the extent of the end-user's medical conditions and the relative rank order of concern of each such condition to the end-user; the end-user's biological and medical profile data such as their biological and physical characteristics comprising height, weight, age, sex, daily or weekly activity levels, etc.; and any of the end-user's nutrition preferences.

### CROSS-REFERENCE TO RELATED APPLICATIONS

This application claims the benefit of priority under 35 U.S.C. § 119(e) of U.S. Provisional Application Ser. No. 63/256,535, filed on Oct. 16, 2022, the content of which is relied upon and incorporated herein by reference in its entirety.

### FIELD

The present disclosure is in the technical field of nutrition, and more particularly, in the technical field of computer-implemented scoring of foods and recipes.

### BACKGROUND

Nutrition plays a significant role in the management of many chronic or health or medical condition(s). Improvements in nutrition of food consumed may lead to better management of chronic or health or medical condition(s) and general health. To improve nutrition of the food consumed, a person would need to have knowledge of the suitability of the foods consumed towards each of one or more of their chronic or health or medical condition(s) as well as the suitability towards general health. Assessing the suitability of a recipe or food to the person in context of one or more chronic or health or medical condition(s) that they suffer from as well as the general health is a challenging task. To manage their one or more chronic or health or medical condition(s), the person, for example, may need to limit the intake of certain nutrients such as sodium in case of hypertension, or increase the intake of certain nutrients such as dietary fibres in case of diabetes mellitus, or keep the intake of potassium within certain limits depending on the stage of chronic kidney disease they might have. Moreover, a recipe or food prepared following the steps of a recipe (henceforth referred to simply as ‘recipe’) if consumed in large quantities, may not be suitable for the person even though the recipe or food is suitable to the person in smaller quantities. Such assessments of suitability of the quantity, in terms of servings or weight or volume, of the recipe or food to the person in the context of their chronic or health or medical condition(s) or general health is also challenging.

A large section of the population has more than one chronic or health or medical condition(s). Having more than one chronic or health or medical condition(s) is also known in medical literature as comorbidity. Comorbidity is defined as the presence of one or more additional chronic or health or medical condition(s) occurring alongside the primary condition(s). For example, a person who has type 2 diabetes may also have dyslipidemia (including hypercholesterolemia) or a person with CKD (chronic kidney disease) stage 4 may also have hypertension as well as type 2 diabetes. When a person suffers from more than one chronic or health or medical condition(s), the management of diet, which includes the foods and recipes consumed, becomes even more challenging because of the combination of limitations due to each of their chronic or health or medical condition(s) as well as limitations pertaining to general health.

Existing computer implemented systems for evaluating various nutritional aspects of a food or recipe rely on nutritional indexes to provide nutritional advise to users, but do not allow the user to override the settings. Such systems, for example, do not allow the user to set nutrient limits that override the default nutrient limits for their medical conditions, do not allow the user to set independent weights to one or more medical conditions to indicate the relative importance of the condition to the user, do not score the food or recipe at various amounts of servings, weights, or volume, do not score the food or recipe in the context of a specific medical condition or in the context of a plurality of medical conditions, and/or do not communicate a script to the user for understanding the scoring. And some existing systems require the input of a food log identifying foods previously consumed by the user. Accordingly, there remains a need for an improved system for scoring foods and recipes that do not require knowledge of prior consumption of foods.

### SUMMARY

In various embodiments, a computer implemented method for scoring a food or recipe for its suitability to one or more medical conditions inputted by a user of an application program configured to carry out the method, comprising: receiving, from a user computing device, a user-generated query for the food or recipe, wherein the food or recipe comprises one or more food components, wherein each respective food component comprises one or more nutrients; retrieving, from a database of the application program in communication with the user computing device, the one or more nutrients for each respective food component and an amount of each respective nutrient; determining a cumulative amount of each nutrient for the food or recipe; determining a per serving amount of each nutrient for the food or recipe; receiving, from the user computing device, the one or more medical conditions inputted by the user; retrieving, from the database of the application program in communication with the user computing device, a default set of nutrient preferences for each respective medical condition, wherein the default set of nutrient preferences comprises a recommended maximum amount for one or more medical condition-specific nutrients; receiving, from the user computing device, a confirmation or modification of the default set of nutrient preferences for each respective medical condition, wherein the modification is configured to override the default set of nutrient preferences for one or more nutrients; determining, for each respective medical condition, a first score indicating the suitability of the food or recipe for the user; and transmitting, to the display of the user computing device, the first score.

In some embodiments, the receiving, from the user computing device, the one or more medical conditions inputted by the user comprises a plurality of medical conditions.

In some embodiments, each of the plurality of medical conditions inputted by the user is assessed a first initial weight of relative importance by the application program.

In some embodiments, the computer implemented method further comprises receiving, from the user computing device, a confirmation or modification of the assessed first initial weight of relative importance for each respective medical condition, wherein the modification is configured to override the first initial weight assessed by the application program.

In some embodiments, the first initial weight of relative importance for each of the plurality of medical conditions inputted by the user is equal.

In some embodiments, the receiving, from the user computing device, a confirmation or modification of the assessed first initial weight of relative importance for each respective medical condition is a modification; and wherein the modification assesses a first user-specific weight of relative importance for each of the plurality of medical conditions and the respective first user-specific weights are not equal.

In some embodiments, the computer implemented method further comprises determining, for all the one or more medical conditions, a second score indicating the suitability of the food or recipe for the user; and transmitting, to the display of the user computing device, the second score.

In some embodiments, the default set of nutrient preferences for at least one of the one or more respective medical conditions comprises a plurality of medical condition-specific nutrients, and wherein each of the plurality of medical condition-specific nutrients is assessed a second initial weight of relative importance by the application program.

In some embodiments, the receiving, from the user computing device, a confirmation or modification of the default set of nutrient preferences for each respective medical condition is a modification; and wherein the modification assesses a second user-specific weight of relative importance for each of the plurality of medical condition-specific nutrients.

In some embodiments, the method further comprises receiving, from a user computing device, a modification of the number of servings for the food or recipe; and determining, for each respective medical condition, the first score indicating the suitability of the food or recipe for the user based on the modified number of servings.

In various embodiments, a computer program product comprising a non-transitory computer readable storage medium having program instructions embodied therewith, the program instructions executable by a computer processor to cause the computer processor to perform a method, comprising: providing an application program for carrying out the method; receiving, from a user computing device, a user-generated query for the food or recipe, wherein the food or recipe comprises one or more food components, wherein each respective food component comprises one or more nutrients; retrieving, from a database of the application program in communication with the user computing device, the one or more nutrients for each respective food component and an amount of each respective nutrient; determining a cumulative amount of each nutrient for the food or recipe; determining a per serving amount of each nutrient for the food or recipe; receiving, from the user computing device, the one or more medical conditions inputted by the user; retrieving, from the database of the application program in communication with the user computing device, a default set of nutrient preferences for each respective medical condition, wherein the default set of nutrient preferences comprises a recommended maximum amount for one or more medical condition-specific nutrients; receiving, from the user computing device, a confirmation or modification of the default set of nutrient preferences for each respective medical condition, wherein the modification is configured to override the default set of nutrient preferences for one or more nutrients; determining, for each respective medical condition, a first score indicating the suitability of the food or recipe for the user; and transmitting, to the display of the user computing device, the first score.

In some embodiments, the receiving, from the user computing device, the one or more medical conditions inputted by the user comprises a plurality of medical conditions.

In some embodiments, the each of the plurality of medical conditions inputted by the user is assessed a first initial weight of relative importance by the application program.

In some embodiments, the computer program product further comprises receiving, from the user computing device, a confirmation or modification of the assessed first initial weight of relative importance for each respective medical condition, wherein the modification is configured to override the first initial weight assessed by the application program.

In some embodiments, the first initial weight of relative importance for each of the plurality of medical conditions inputted by the user is equal.

In some embodiments, the receiving, from the user computing device, a confirmation or modification of the assessed first initial weight of relative importance for each respective medical condition is a modification; and wherein the modification assesses a first user-specific weight of relative importance for each of the plurality of medical conditions and the respective first user-specific weights are not equal.

In some embodiments, the computer program further comprises determining, for all the one or more medical conditions, a second score indicating the suitability of the food or recipe for the user; and transmitting, to the display of the user computing device, the second score.

In some embodiments, the default set of nutrient preferences for at least one of the one or more respective medical conditions comprises a plurality of medical condition-specific nutrients, and wherein each of the plurality of medical condition-specific nutrients is assessed a second initial weight of relative importance by the application program.

In some embodiments, the receiving, from the user computing device, a confirmation or modification of the default set of nutrient preferences for each respective medical condition is a modification; and wherein the modification assesses a second user-specific weight of relative importance for each of the plurality of medical condition-specific nutrients.

In some embodiments, the computer program product further comprises receiving, from a user computing device, a modification of the number of servings for the food or recipe; and determining, for each respective medical condition, the first score indicating the suitability of the food or recipe for the user based on the modified number of servings.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein and, together with the description, explain the principles and operations of the claimed subject matter.

### BRIEF DESCRIPTION OF THE DRAWINGS

A complete understanding of the present embodiments and the advantages and features thereof will be more readily understood by reference to the following detailed description, appended claims, and accompanying drawings, wherein:

FIG. 1 is a block diagram of a client-server system, in accordance with embodiments described herein;

FIG. 2 is a block diagram of a peer-to-peer system, in accordance with embodiments described herein;

FIG. 3 is a block diagram of a standalone system, in accordance with embodiments described herein;

FIG. 4 is a block diagram of a client-side implementation, in accordance with embodiments described herein;

FIG. 5 is a block diagram of a server-side implementation, in accordance with embodiments described herein;

FIG. 6 is a flowchart illustrating a method of scoring of a recipe or a food, in accordance with embodiments described herein;

FIG. 7 is an example of a table of records pertaining to nutrient-preferences of users, in accordance with embodiments described herein;

FIG. 8 is an example of a table comprising records pertaining to various chronic or health or medical conditions for a user, in accordance with embodiments described herein;

FIG. 9 is an example of a table comprising configurations of nutrients that need to be considered when scoring recipes or foods, in accordance with embodiments described herein;

FIG. 10 is an example of a table comprising records pertaining to scores for ranges of percentage daily values of nutrients, in accordance with embodiments described herein;

FIG. 11 is an example of a table comprising records pertaining to one or more nutrient-preferences for various chronic or health or medical condition(s), in accordance with embodiments described herein;

FIG. 12 is a partial record in JSON (JavaScript Object Notation) format illustrating an example of a document or record depicting a user's preferences on limits of consumption of certain nutrients, in accordance with embodiments described herein;

FIG. 13A is a partial record in JSON (JavaScript Object Notation) format illustrating an example of a document or record depicting a user's list of chronic or health or medical conditions along with the rank the user may assign to the importance of that condition to them, in accordance with embodiments described herein;

FIG. 13B is a partial record in JSON (JavaScript Object Notation) format illustrating an example of a document or record depicting a system-wide standard or system-wide universal list of chronic or health or medical conditions with a rank and weight assigned to each chronic or health or medical condition, in accordance with embodiments described herein;

FIG. 14 is a partial record in JSON (JavaScript Object Notation) format illustrating an example of a document or record depicting a list of nutrients along with the amount of the nutrients that comprise an ingredient, in accordance with embodiments described herein;

FIG. 15 is a partial record in JSON (JavaScript Object Notation) format illustrating an example of a document or record depicting a list of nutrient-preferences for various chronic or health or medical condition(s) including ‘general health’, in accordance with embodiments described herein;

FIG. 16 is a partial record in JSON (JavaScript Object Notation) format illustrating an example of a code snippet of a document or record depicting a mapping of a subset range of overall score to a color code and letter grade, in accordance with embodiments described herein;

FIG. 17 is a partial record in JSON (JavaScript Object Notation) format illustrating an example of a code snippet of a document or record depicting a mapping of a subset range of score for a chronic or health or medical condition(s) to a color code and letter grade, in accordance with embodiments described herein;

FIG. 18 is a partial record in JSON (JavaScript Object Notation) format illustrating an example of a code snippet of a document or record depicting a list of weights for relevant nutrients for each combination of various chronic or health or medical condition(s), including ‘general health,’ that target users may be interested in, in accordance with embodiments described herein;

FIG. 19 illustrates an example of a code snippet of an API response in JSON (JavaScript Object Notation) format for a scoring request for scoring of a certain amount of a recipe or food for a user who may be interested in a set of chronic or health or medical conditions, in accordance with embodiments described herein;

FIG. 20 is a partial record in JSON (JavaScript Object Notation) format illustrating an example of a code snippet of a document or record depicting a weight for each condition in various combinations of various chronic or health or medical conditions, including ‘general health,’ that the target user may be interested in, in accordance with embodiments described herein;

FIG. 21 illustrates an example of a code snippet in JSON (JavaScript Object Notation) format illustrating an example configuration that may be used to generate messages for certain nutrients based on the score for the nutrient for a recipe or food at a certain amount or serving for a target user, in accordance with embodiments described herein;

FIG. 22 illustrates an example of a code snippet in JSON (JavaScript Object Notation) format illustrating an example configuration that may be used to generate messages for the overall score of a recipe or food for the target user, in accordance with embodiments described herein;

FIG. 23 illustrates the interpretation of the terminology [percentage1, percentage2) for a given value v, in accordance with embodiments described herein;

FIG. 24 is a part of the flowchart illustrating an example of the steps taken by the method of scoring a recipe or a food, in accordance with embodiments described herein;

FIG. 25 is a continuation of the flowchart in FIG. 24, in accordance with embodiments described herein;

FIG. 26 shows the details of the step 422 in FIG. 25, in accordance with embodiments described herein;

FIG. 27 shows one way the step 526 in FIG. 26 can be achieved, in accordance with embodiments described herein;

FIG. 28 shows another way the step 526 in FIG. 26 can be achieved, in accordance with embodiments described herein;

FIG. 29 shows another way the step 526 in FIG. 26 can be achieved, in accordance with embodiments described herein;

FIG. 30 is an example user interface for displaying to the user the scores for a selected amount of a recipe or food, in accordance with embodiments described herein;

FIG. 31 is an example user interface for displaying to the user the scores for a selected amount of a recipe or food different from that selected in FIG. 30, in accordance with embodiments described herein;

FIG. 32 is an example user interface for displaying to the user the scores for a selected amount of a recipe or food different from that selected in both FIG. 30 and FIG. 31, in accordance with embodiments described herein;

FIG. 33 is an example user interface in which a target user may set and save their medical and biological information, in accordance with embodiments described herein; and

FIG. 34 is an example user interface in which a target user may set and save their preferences for limits on consumption of certain nutrients per unit of time duration, usually a day, in accordance with embodiments described herein.

The drawings are not necessarily to scale, and certain features and certain views of the drawings may be shown exaggerated in scale or in schematic in the interest of clarity and conciseness.

### DETAILED DESCRIPTION

Reference will now be made in detail to the present preferred embodiment(s), and examples of which is/are illustrated in the accompanying drawings. Whenever possible, the same reference numerals will be used throughout the drawings to refer to the same or like parts. Any specific details of the embodiments are used for demonstration purposes only, and no unnecessary limitations or inferences are to be understood therefrom.

Before describing in detail exemplary embodiments, it is noted the embodiments reside primarily in combinations of components and procedures related to the system. Thus, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In various embodiments, computer-implemented methods and systems for scoring the suitability of a recipe or food to a target user or target subject who has or is interested in one or more chronic or health or medical condition(s), or general health, are provided.

As used herein, the term “medical condition” refers to an illness, ailment, disorder, or disease that affects a person. Examples of medical conditions include chronic kidney disease (CKD) (stages 1, 2, 3, 4, 5), end stage renal disease (ESRD), diabetes mellitus (type-1, type-2, LADA), hypertension, dyslipidemia, polycystic ovarian syndrome, gastrointestinal conditions, etc.

As used herein, the term “score” includes a numeric value, a letter grade, a color, a message text, or any combination thereof. In some embodiments, the score is based on any combination of one or more of: (a) the nutrient content of the food or a food created following the steps of the recipe, (b) the chronic or health or medical condition(s) or general health that the target user may be interested in, (c) the extent of the target user's chronic or health or medical condition(s) and the relative rank order of concern of each such condition to the target user, (d) the target user's biological and medical profile data such as their biological and physical characteristics comprising height, weight, age, sex, daily or weekly activity levels, etc., (e) any of the target user's nutrition preferences. The methods and systems presents to the user a representation of the score of the recipe or food for each of one or more of the chronic or health or medical condition(s) or for general health or a combination thereof. The methods and systems also presents to the user messages regarding the suitability of the food or recipe to their one or more chronic or health or medical condition(s), or general health.

In the following, various embodiments are presented for methods and systems for scoring of foods and recipes for a person who is a target user or individually for a set of persons who are the target users. As used herein, the term ‘person,’ ‘user,’ and ‘subject’ are used interchangeably. All these three: ‘person,’ ‘user,’ and ‘subject’ refer to the target user to whom the methods and systems are addressed. The target user may be the actual user (“end-user”) to whom the methods and systems are addressed. The target user may be an archetypal user who does not exist in reality but is a representative of a set of actual one or more target users that have various characteristics similar to the archetypal user, such characteristics comprising one or more of the following: age, sex, race, daily activity level, BMI (Body Mass Index,) chronic or health or medical condition(s), and preferences on limits on daily consumption of certain nutrients, etc.

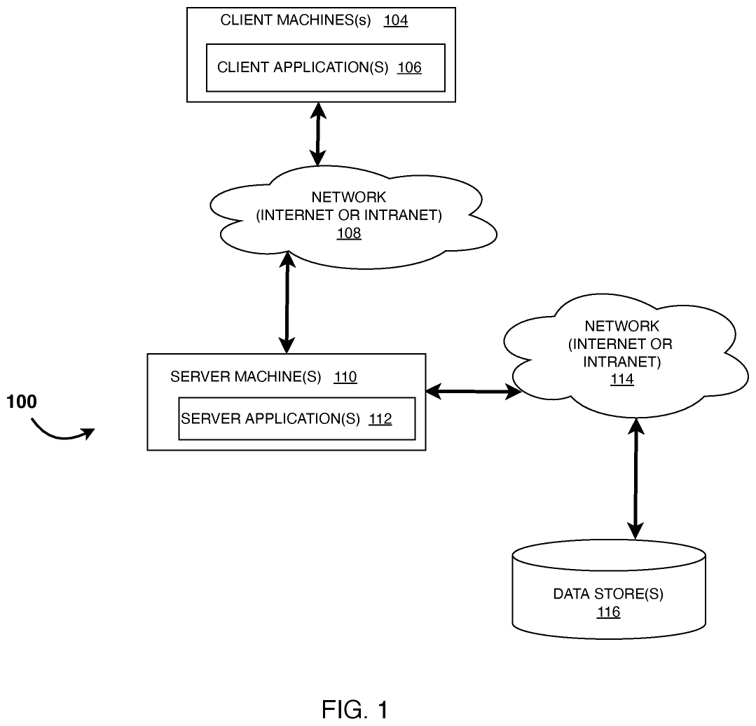

FIG. 1 shows a network diagram illustrating an example of a system 100 within which various embodiments may be deployed. The system 100 comprises one or more server machines 110, which has/have one or more server applications 112 provide server-side functionality. The server-side functionality may be made available via a computer network 108 to one or more client applications 106 running on one or more client machines 104. The client machines may include mobile devices or non-mobile devices. Mobile devices are those devices that are capable of being carried around. Examples of such mobile devices include smartphones, universal product code (UPC) scanners, barcode scanners, QR code scanners, tablets, laptops, specialized medical devices etc. Non-mobile devices are those devices that may not be capable of being carried around. Examples of non-mobile devices include desktop computers and specialized medical devices.

The server application(s) 112, which may run inside server machine(s) 110, may provide services to clients or users of the service. The server machine(s) 110 may be physical machine(s) or virtual machine(s). These services may include the subject matter of this disclosure. In some embodiments, some of the functionality of the server application(s) 112 may be a part of the client application(s) 106. In some embodiments, some of the functionality of the client application(s) 106 may be a part of the server application(s) 112. In various embodiments, the server application(s) 112 may include one or more Application Programming Interface (API) server(s) or any computer server(s) to provide a programmatic data interface for one or more services on one or more application server(s). Those skilled in the art will readily understand that some of the functionality in the server application(s) 112 may be performed in the client application(s) 106 while some of the functionality in the client application(s) 106 may be performed in the server application(s) 112. The server machine(s) 110 may be connected to one or more data store(s) 116. The server machine(s) 110 and the data stores(s) 116 may be connected directly or may be connected through a computer network 114. The computer networks 108 and 114 may be a part of an intranet or a part of the internet. While FIG. 1 shows a client-server architecture 100 within which various embodiments of the methods and systems described here may be implemented, this disclosure is not limited to such an architecture. Various embodiments of the methods and systems may be arranged in a peer-to-peer 120 or a distributed architecture or as a standalone architecture 140 with or without any network or data interface.

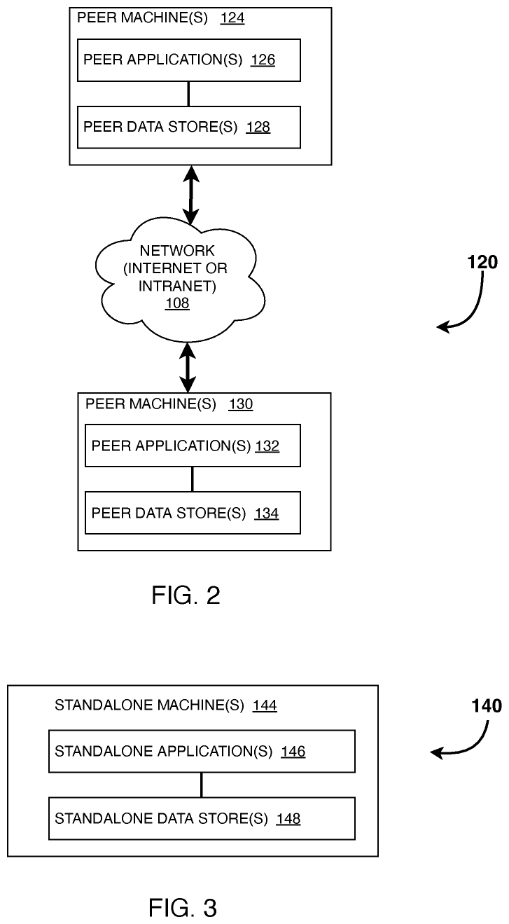

FIG. 2 shows a peer-to-peer architecture that illustrates another example of a system 120 within which various embodiments may be deployed. The system 120 comprises at least one peer machine 124 (130 is for illustration of the peer-to-peer nature of this architecture), each of which have peer application(s) 126 (and 132) as well as peer data store(s) 128 (and 134). Both the client-side and server-side functionality may be provided in the peer application or some functionality may be shared between the peer applications on separate peer machines.

FIG. 3 shows a standalone architecture which illustrates another example of a system 140 within which various embodiments may be deployed. In this architecture, the standalone machine(s) 144, the standalone application(s) 146 or the standalone data store(s) 148 may or may not be attached to a network, whether a part of an intranet or a part of the internet. The system 140 comprises at least one standalone machine(s) 144. The standalone machine(s) comprise standalone application(s) 146 which connect with standalone data store(s) 148. Both the client-side and server-side functionality may be provided in the standalone application(s) 146 or some functionality may be shared with a remote machine(s) if the standalone machine(s) 144 or standalone application(s) 146 or the standalone data store(s) 148 is/are attached to a network.

As discussed herein, the methods and systems will be in the context of the client-server architecture 100 unless components of the peer-to-peer architecture, i.e., 124, 126, 128, 130, 132 or 134 or those of the standalone architecture, i.e., 144 or 146 or 148 are explicitly mentioned. Those skilled in the art will readily understand that the embodiments described for a client-server architecture may easily be applied to the distributed or peer-to-peer architecture 120 or to the standalone architecture 140 or various combinations of these or various other architectures not illustrated here.

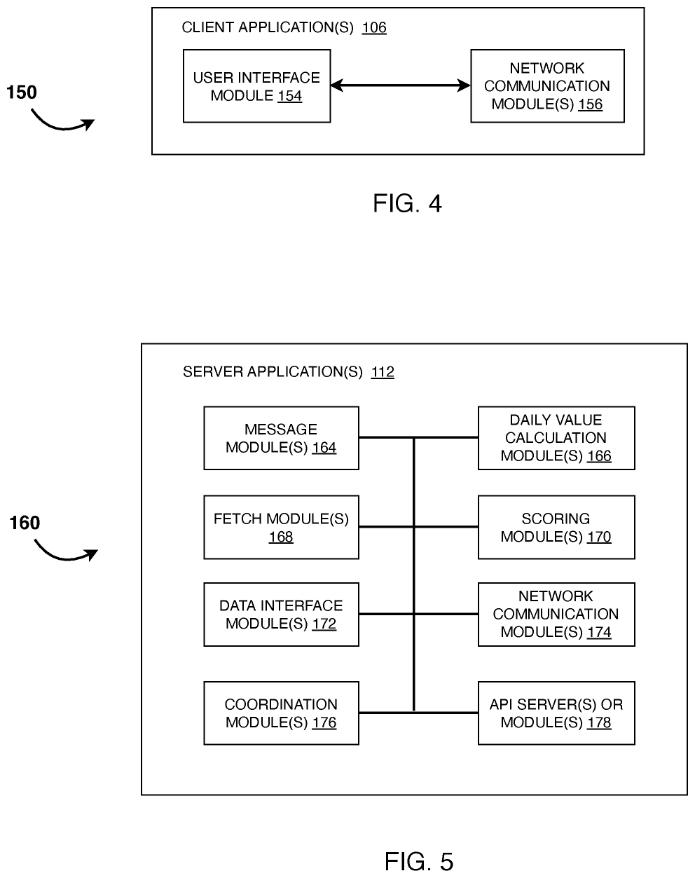

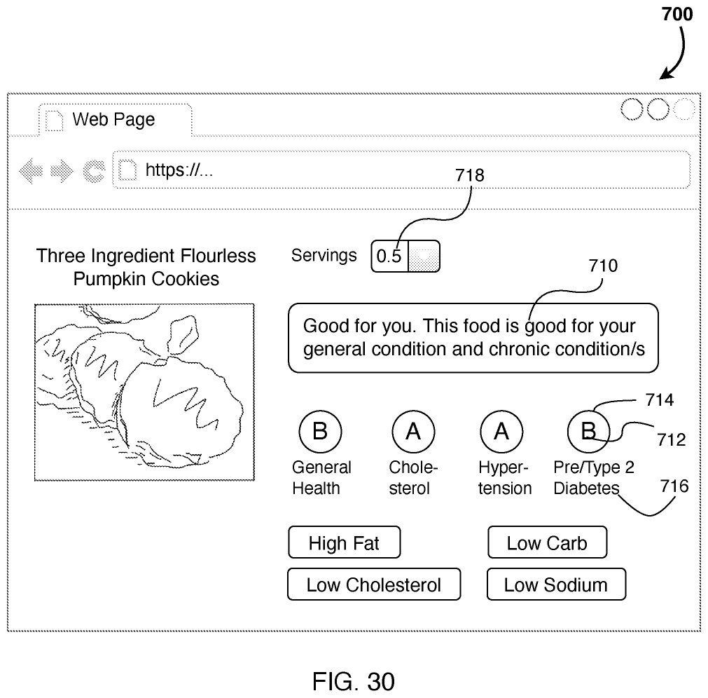

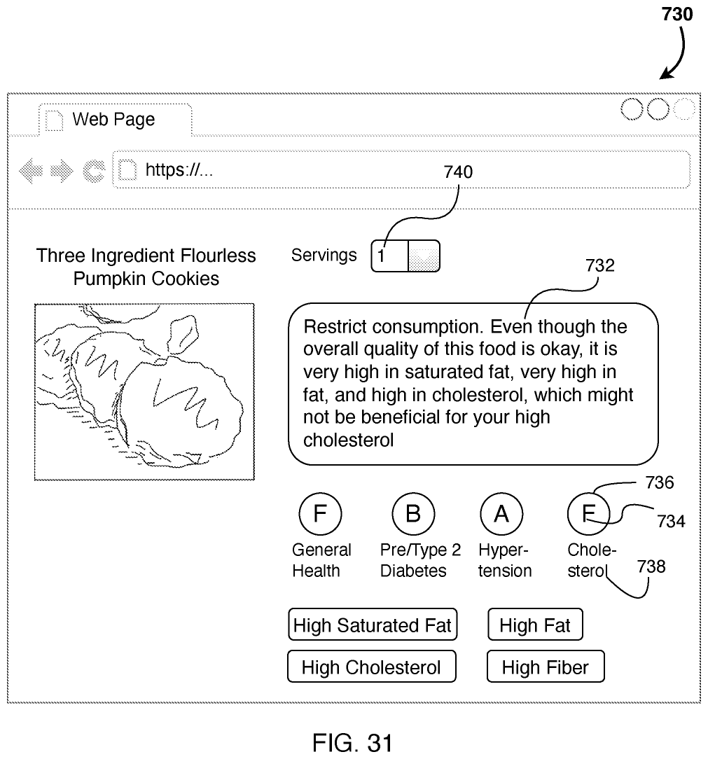

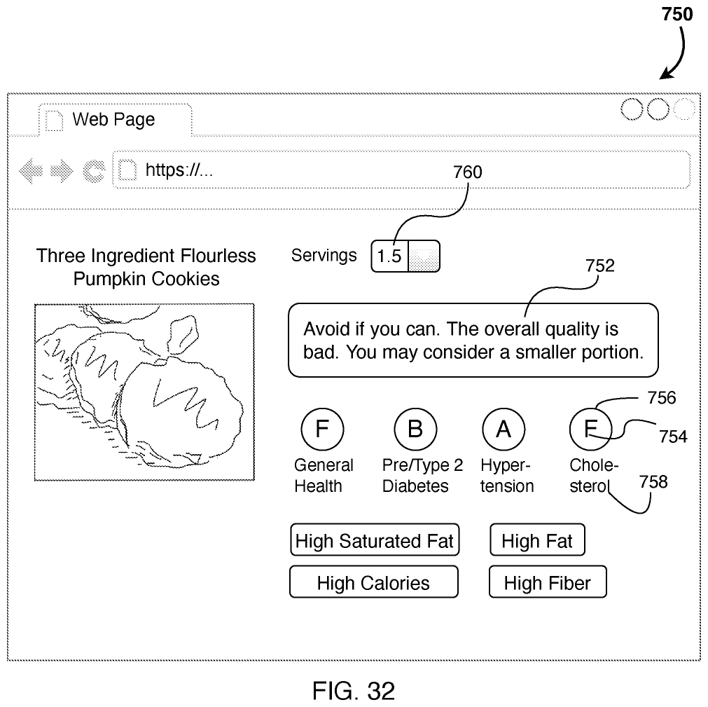

The block diagram 150 in FIG. 4 illustrates example modules of the client application(s) 106. The user interface module 154 may be configured to allow the user to interact with the user interface of the client application(s) 106. For example, the user interface module may be configured to present to the user at least some or all of the example user interfaces 700, 730 and 750 in FIG. 30, FIG. 31, and FIG. 32. The network communication module(s) 174 may be used to communicate with the server application(s) 112 or peer application(s) 126, 132 or peer data store(s) 128, 134. The network communication module(s) 156 may be configured to allow the user interface module 154 to connect to the network 108.

The block diagram 160 in FIG. 5 illustrates example modules of the server application(s) 112. The daily value calculations module(s) 166 may be configured to calculate the daily values of various nutrients for one or more users of the client application(s) 106. The scoring module(s) 170 may be used to score recipes or foods for users of client application(s) 106 who may be interested in certain chronic or health or medical condition(s) including but not limited to diabetes (type 1 and type 2), hypertension, dyslipidemia, chronic kidney disease at stages 1, 2, 3, 4, 5, end stage renal disease (ESRD), polycystic ovary syndrome (PCOS), gastrointestinal conditions, etc. The fetch module(s) 168 may be configured to allow the various example modules in the server application(s) 112 to collect or collate or gather or process or massage data to make the data easier to further process. The data interface module(s) 172 may be configured to allow the various example modules in the server application(s) 112 to interact with or exchange data with the data store(s) 116, 128, 134, or 148. The message module(s) 164 may be configured to create messages for the user of the client application(s) 106 based either on the scoring of a recipe or food or on the scoring of relevant nutrients or on the user's nutrition or other preferences or on chronic or health or medical condition(s) that the user might be interested in. The network communication module(s) 174 may be configured to communicate the scores of the recipes or foods or the messages to the client application(s) 106. The coordination module(s) 176 may be configured to coordinate the processing of a request to score a recipe or food for a target user who may be interested in one or more of general health or chronic or health or medical condition(s). In this disclosure, such requests may sometimes be referred to as the ‘scoring request’ unless otherwise specified. A ‘scoring request’ may occur independently as an independent request or it may be a part of a bigger request. The server application(s) may process the scoring request as such regardless of whether it is an independent request or a part of a bigger request which may encompass the scoring request. The coordination module(s) 176 may, with the help of the network communication module(s) 174, receive a scoring request, perform the scoring of the recipe(s) or food(s) in the scoring request for the target user(s) within appropriate contexts including the condition(s) for the target user(s), generate messages some or all of which may correspond to the score(s) or condition(s) etc., and send the response back to the requester. In achieving the goal of processing the scoring request, the coordination module(s) may make use of any or all of the other module(s) in 160.

The API server(s) or module(s) 178 may be configured to: (a) receive scoring requests, (b) process the scoring request with the help of the coordination module(s), and (c) send back a response which may comprise the scores and messages for one or more amounts of the recipe or food for one or more of general health or chronic or health or medical condition(s) that a target user may be interested in.

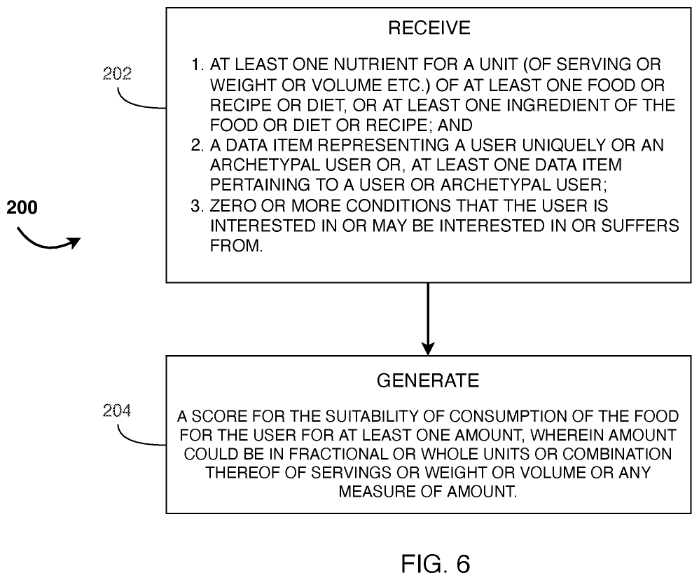

FIG. 6 is a flowchart illustrating example operations of a method 200 of generating scores for at least one recipe or food for a target user who may be interested in general health or at least one chronic or health or medical condition(s) or a combination thereof. In various embodiments, method 200 may be implemented by one or more modules of the client application(s) 106 or the server application(s) 112 or the peer application(s) 126 (or 132) or the standalone application(s) 146. In various embodiments, some parts of the method 200 may be implemented by one or more modules of the client application(s) 106 while some of the other parts may be implemented by one or modules of the server application(s) 112. In various other embodiments, where the system is arranged in a peer-to-peer or distributed architecture 120, the method 200 may be implemented by one or more modules of the peer application(s) 126 (or 132) and peer data store(s) 128 (or 134) on one or more of the peer machine(s) 124 (or 130). For example, in a peer-to-peer arrangement 120, the method 200 may be implemented in at least one peer machine 124 or (130.) As another example, in a standalone arrangement 140, the method 200 may be implemented in the standalone machine 144 as a part of the standalone application 146 and the standalone data store 148.

At operation 202, the network communications module 174 receives the scoring request to score at least one unit of at least one recipe or food for a user. For a standalone architecture, the scoring request may be received directly by the coordination module(s) 176. It will be readily apparent to those skilled in the art that while the following is described for the case where the scoring request is for a single user, the method 200 may easily be extended to handle the case of a scoring request containing a set of several sub-requests for a plurality of users where each sub-request is similar to the scoring request described.

The scoring request may include a list of ingredients used to create the food from the recipe. Each ingredient may have associated with it an amount in terms of weight or volume of the ingredient. For example, 1 cup of wheat flour.

The scoring request may also include the yield of the recipe. The yield represents the amount of the food that is created by following the steps of the recipe. The yield may be specified in terms of weight, e.g., 100, grams or in terms of volume, e.g., 120 ml, or in terms of servings, e.g., 4 servings.

The scoring request may include at least one nutrient of the recipe or food along with the amount of the nutrient, e.g., 72 grams of total carbohydrates, 231 mg of sodium.

The scoring request may additionally include at least one identifier, where each identifier uniquely identifies a user for which the scoring of the recipe or food is requested.

As used herein, an identifier that may uniquely refer to a user or a subject may be referred to as userid. As an alternative to including the userid, the scoring request may include a collection of data items pertaining to at least one user, where at least one data item pertaining to a user specifying partly or wholly the user's profile data such as their biological and physical characteristics such as height, weight, age, sex, race, waist measurement, chest measurement, wrist measurement, daily or weekly activity levels, etc. Such collection of at least one data item pertaining to a user may be used to represent an archetypal user where such a user may not exist in real life but may be representative of a group of people who may be similar to such an archetypal user in aspects specified by way of the data items. Such an archetypal user may still be referred to as a userid.

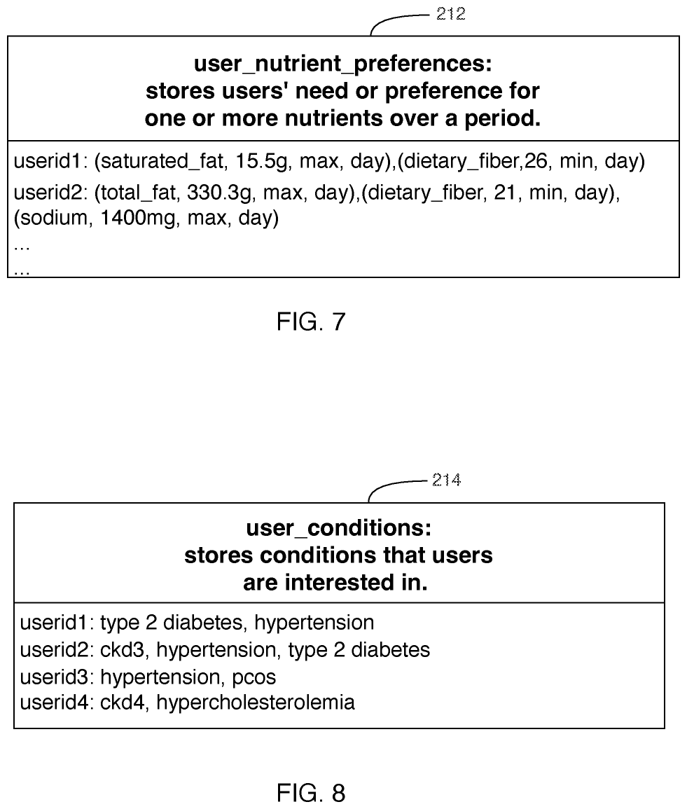

As an alternative to including the userid, the scoring request may include a list of one or more nutrient-preferences. Nutrient-preferences is a list consisting of restricting values for a set of nutrients. Examples of nutrient-preferences for a target user are shown in table 212 and may include the daily needs of various nutrients for a target user. The contents of the data shown in table 212 in FIG. 7 may be stored in the data store(s) 116 or the peer data store(s) 128 (or 134) or the standalone data store(s) 148. In FIG. 7, a nutrient-preference comprises: (a) the name of the nutrient; (b) a limit value which may be an absolute value or a percentage of the total calorie needs of the target user or an absolute value dependent on certain characteristics of the target user; (c) a maximum (max) or a minimum (min) designation for the type of said limit value; (d) the period over which the limit applies, usually, a day.

The target user may want to restrict the consumption of the nutrients specified in the list to the values specified over a period of time, usually a day. The restriction may be a minimum restriction or a maximum restriction. A minimum restriction refers to a restriction where the target user would like to consume, approximately, at least the specified amount over a certain period of time. A maximum restriction refers to a restriction where the target user would like to roughly consume, approximately, at most the specified amount over a certain period of time. The target user may want the restriction on consumption of the nutrients because of one or more of the following: (a) the target user may be concerned about or be suffering from one or more chronic or health or medical condition(s), (b) the target user may want to improve or be concerned about their general health, (c) the target user may want to lose or gain weight, (d) the target user may want to avoid the development of one or more chronic or health or medical condition(s), or (d) for any other reason. Such nutrient-preferences may be provided in the incoming scoring request in step 202 or may be obtained by the application(s) 112 (or 106 or 126 or 132 or 146) from the data store(s) 116 in the case of the client server architecture or the data store(s) 128 (or 134) in the case of a peer-to-peer or distributed architecture or the data store(s) 148 in the case of a standalone architecture.

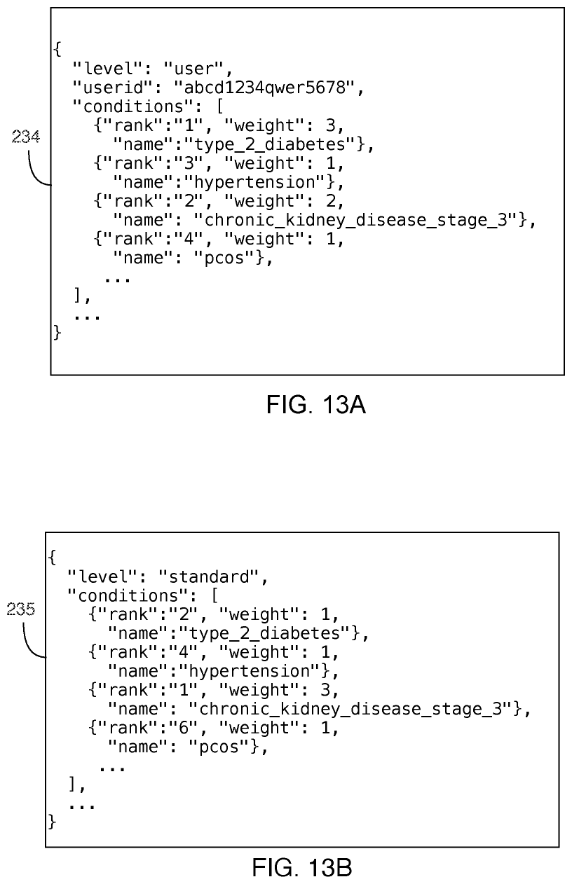

The scoring request may additionally include names of zero or more chronic or health or medical condition(s) that the target user may be interested in. The scoring request may additionally include ‘general health’ if the target user is interested in their general health. In various embodiments, in the case where zero chronic or health or medical condition(s) are specified in the scoring request or the target user for whom the scoring request has been made has no chronic or health or medical condition(s) known to the system, the system may assume that the scoring request is for ‘general health.’ The chronic or health or medical condition(s) may include diabetes (type 1 and type 2,) hypertension, dyslipidemia, chronic kidney disease at various levels, polycystic ovary syndrome etc. In various embodiments, the chronic or health or medical condition(s) or the nutrient-preferences may be stored in data store(s) such as 116 or 128 or 134 or 148. An example code snippet of a record 234 of a list of chronic or health or medical condition(s) that a target user may be interested in is illustrated in FIG. 13A. This code snippet may be a part of the scoring request or a part of a data record for the target user. The chronic or health or medical condition(s) that the target user in the scoring request may be interested in may be obtained from such a data source if these condition(s) are not specified in the scoring request.

At operation 204, for the target user specified in the scoring request, the daily value calculations module(s) 166, calculates the daily values of all relevant nutrients, if at least one of all daily values for relevant nutrients are not specified in the scoring request. These daily values are the maximum or minimum amount of each relevant nutrient that the target user may want to or need to consume in a period, usually a day. These daily values are in units of weight or volume, needed to be consumed, usually daily, by the target user assuming that the user has the chronic or health or medical condition(s) that the user may be interested in.

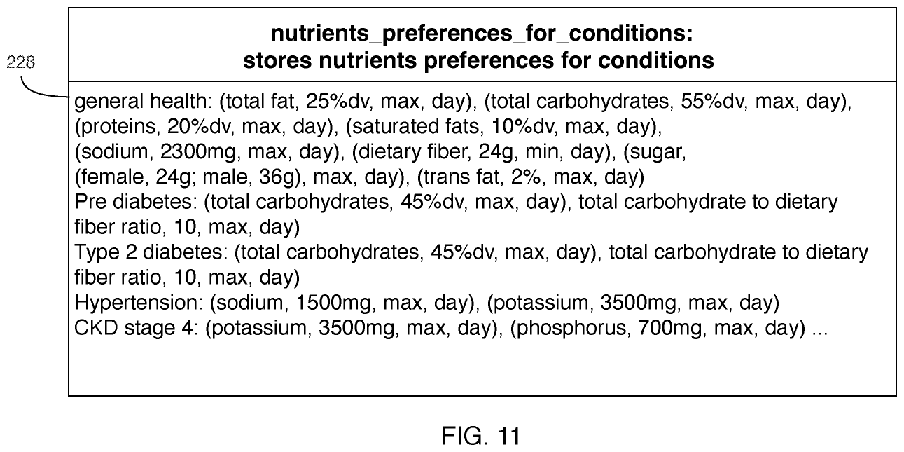

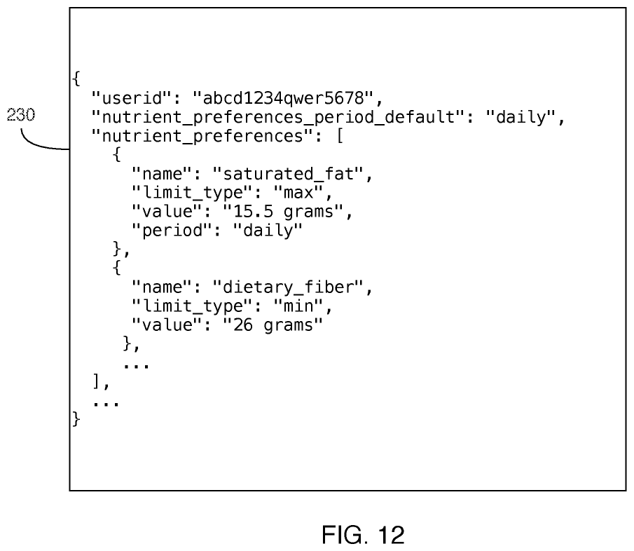

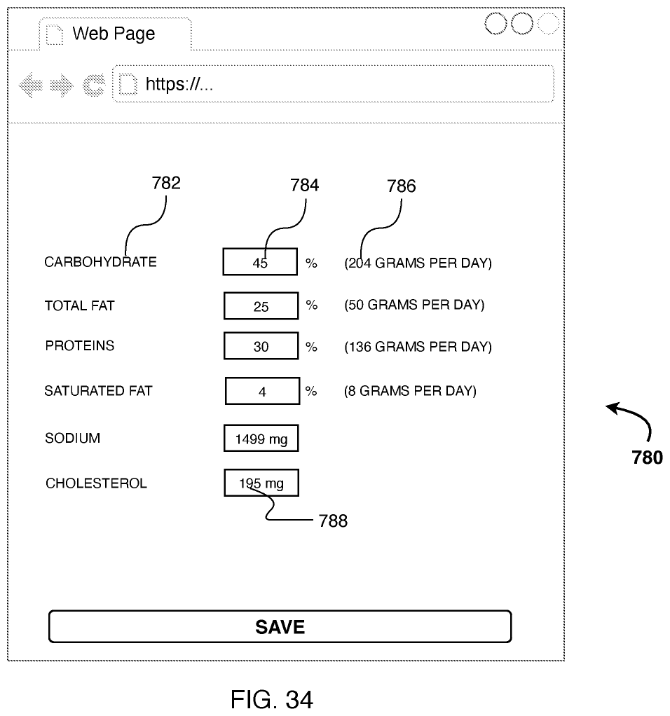

The nutrient-preference(s) of a target user may be stored in the data store(s) 116 or 128 or 134 or 148. An example of such a nutrient-preference record which may be stored in a JSON (JavaScript Object Notation) format is depicted in the code snippet 230 in FIG. 12. This record may reflect the user specifying their nutrient-preference(s) using a user interface similar to the sample user interface 780 is in FIG. 34. The nutrient-preference may also be set by the target user using an API call made to the application(s) 112 (or 106 or 126 or 132 or 146). In case the target user has not specified a nutrient-preference for any nutrient(s) that are relevant to their chronic or health or medical condition(s) or general health, the limit-preferences for those nutrient(s) may be obtained from one or more tables or documents in the data source(s) 116 or 128 or 134 or 148 and these limit-preferences may be based on the chronic or health or medical condition(s) that the user may be interested in. An example of such limit-preferences is shown in table 218 in FIG. 11, which shows a percentage daily values of calorie needs of a user or an absolute value or a value dependent on certain characteristics of the user such as their gender, lactating condition (i.e., whether the target user is lactating or not), pregnancy condition (i.e., whether the target user is pregnant or not), age, etc.

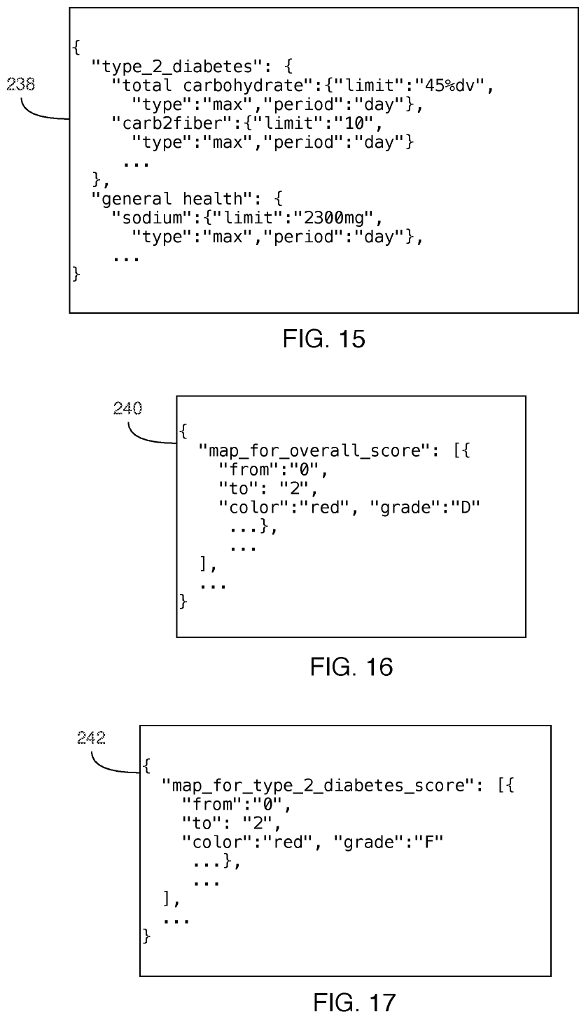

Additionally, nutrient-preferences for various chronic or health or medical condition(s), including ‘general health,’ may also be defined in the system. An example of such nutrient-preferences stored, for example, in one or more tables in a relational database is illustrated in the example table 228 in FIG. 11. An example of such a nutrient-preference record stored in a JSON (JavaScript Object Notation) format is depicted in the code snippet 238 in FIG. 15. Such nutrient-preferences may also be stored in at least one of non-relational database, relational database, file system, memory and any other device capable of storing data.

For consideration in scoring of a recipe or food, if a nutrient is found to be relevant to a target user based on, say, one or more of the target user's chronic or health or medical condition(s) or general health, or based on any other consideration, and the nutrient has been configured by the target user, for instance, by way of a user interface similar to 780, and hence is present in the example record 230 (also shown in example table 212), then the nutrient- preference from the record 230 is considered in scoring of the recipe or food for the target user. In some embodiments, if the target user has expressed a nutrient-preference, then the system may honour it over the nutrient-preference derived from the condition(s) that the target user may be interested in. If, however, the target user has not expressed a nutrient-preference, then the preference for the nutrient is obtained from the example table illustrated in 228. The nutrients that are relevant for a target user may partly or wholly be obtained from example tables 228 and 212. If a nutrient appears for more than one condition(s), then the nutrient-preference of the condition that the target user considers more important is considered. A rank ordering of importance of the various condition(s) may be elicited from the target user and saved in the system in the data store(s) 116 or 128 or 134 or 148. This rank ordering is illustrated as “rank” in the code snippet 234 in FIG. 13A. This rank ordering may be used to identify the nutrient-preference that may be applied to the nutrient in the event of the nutrient being present for more than one condition(s) that the user may be interested in in the following way: if, for a nutrient that is salient for any one or more of the condition(s) that the target user may be interested in, the user has not expressed a nutrient-preference, then the nutrient-preference for that nutrient is obtained from the condition among the condition(s) that the user may be interested in that has the highest rank for the user and for which the nutrient is salient. In said scheme, if the user has not specified any ranking, then a system-wide standard ranking may be used. An example of such a system-wide standard rank ordering of conditions is illustrated in the configuration code snippet 235 in FIG. 13B.

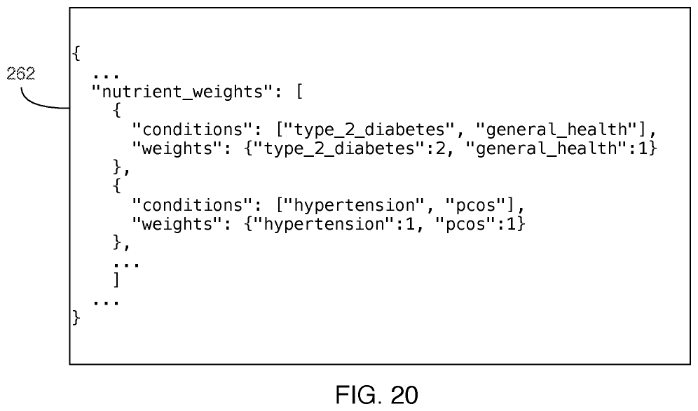

In various embodiments, weights may be assigned to various combinations of one or more of the various chronic or health or medical condition(s). The weights may be elicited from the target user and saved in the system in the data store(s) 116 or 128 or 134 or 148. These weights are illustrated as “weight” in the code snippet 234 in FIG. 13A. If a user has not chosen to assign any weights to the various chronic or health or medical condition(s) that the user is interested in, a set of standard weights as defined in the system may be used. An example of such a definition is illustrated in an example record 262 in FIG. 20. If, however, a user has not chosen to assign any weights to the various chronic or health or medical condition(s) the user is interested in and the system does not have an entry for the combination of the various chronic or health or medical condition(s) the user may be interested in, then, in yet some other embodiments of this disclosure, the weights for the conditions as defined in the example code snippet 235 in FIG. 13B may be used.

The nutrient-preferences illustrated in the example tables are for illustration purpose only. The system may be configured such that the nutrient-preferences may be defined for several chronic and non-chronic health or medical condition(s). For example, if the target user is interested in type 2 diabetes and it will be readily apparent to those skilled in the art that, in various embodiments, the list of nutrients in table 228 can be extended to include other nutrients for any of the chronic or health or medical condition(s) depicted in table 228. In addition, those skilled in the art will readily understand that various embodiments may contain such lists for other chronic or health or medical condition(s) not listed in the example list in table 228. In addition to the nutrients for the chronic or health or medical condition(s) that the user may be interested in, nutrients for general health may also be included in the list of relevant nutrients. The nutrients for general health may include other nutrients that are not presented in the example table 228.

In order to complete its task, the daily value calculations module(s) 166 may need to use the fetch module(s) 168, the data interface module(s) 172 or the network communications module(s) 174, or a combination thereof, to fetch, if needed, details about the target user and details about the one or more chronic or health or medical condition(s) the target user may be interested in.

Once daily value amounts of nutrients for the target user have been calculated by the daily value calculations module(s) 166, the scoring module(s) 170 fetches the amounts for each nutrient present in the amount of the recipe or food specified in the scoring request. The amount of the recipe or food to be considered for scoring may be specified in the scoring request. Alternatively, a standard amount for the recipe or food may be configured in the system for a given target user such that if the scoring request does not have an amount specified for the recipe or food, the scoring module(s) 170 may fetch this standard value for the requested amount from one of the data store(s) 116 or 128 or 134 or 148. Examples of the standard amount may be 1½ a serving or 110 grams, 150 ml, or ½ serving etc.

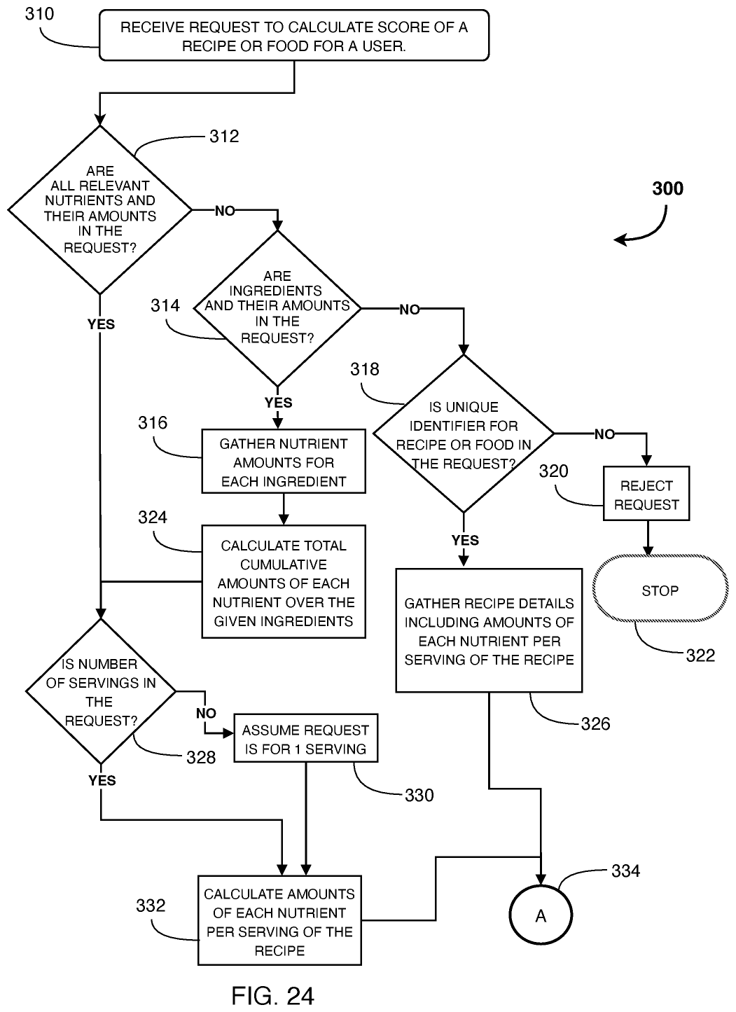

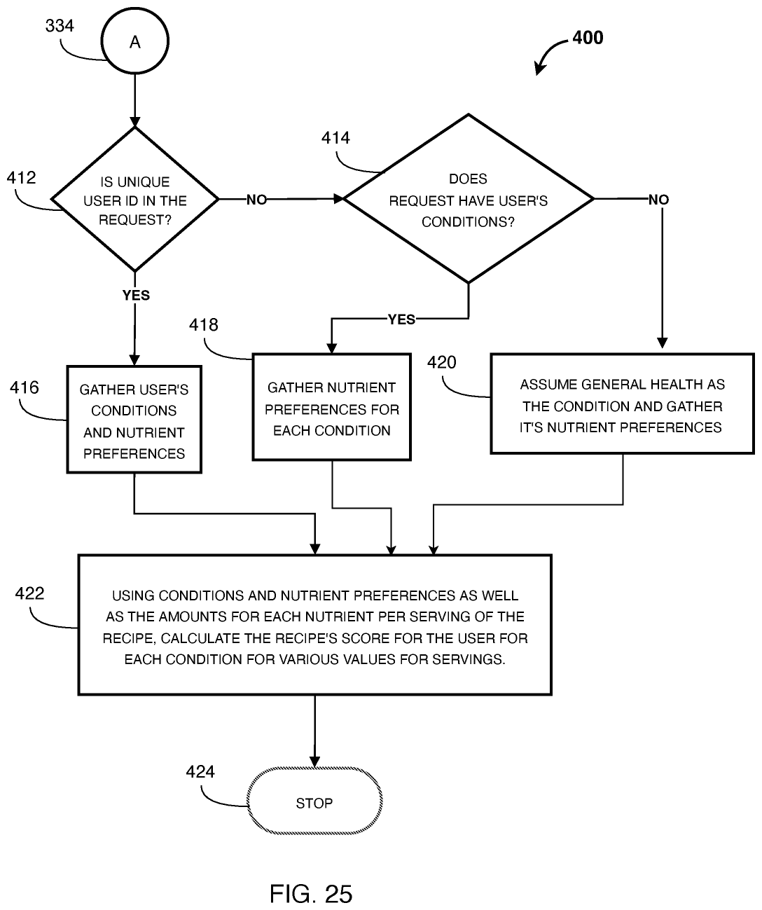

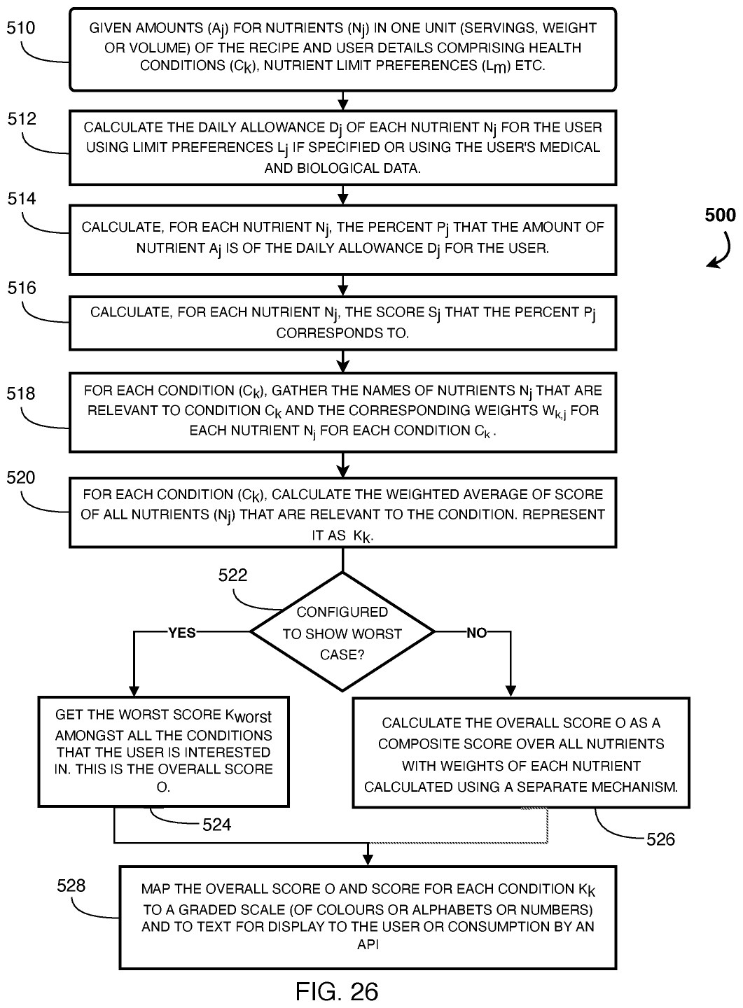

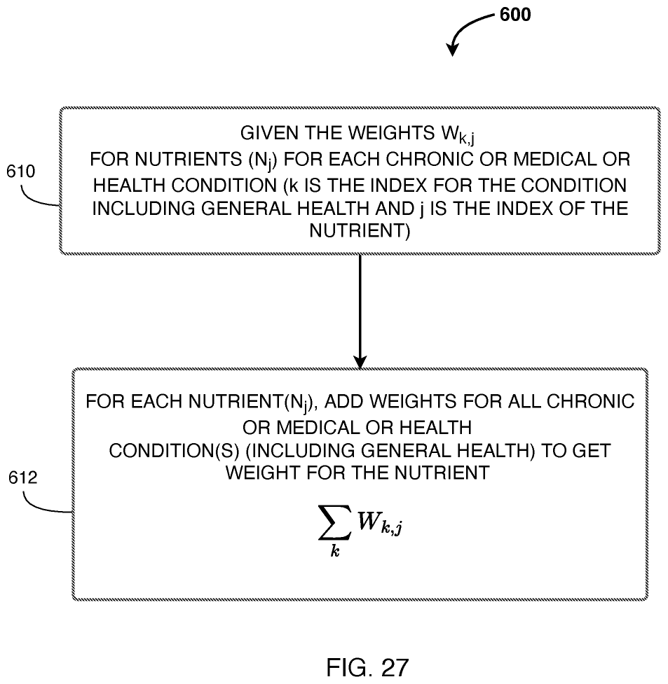

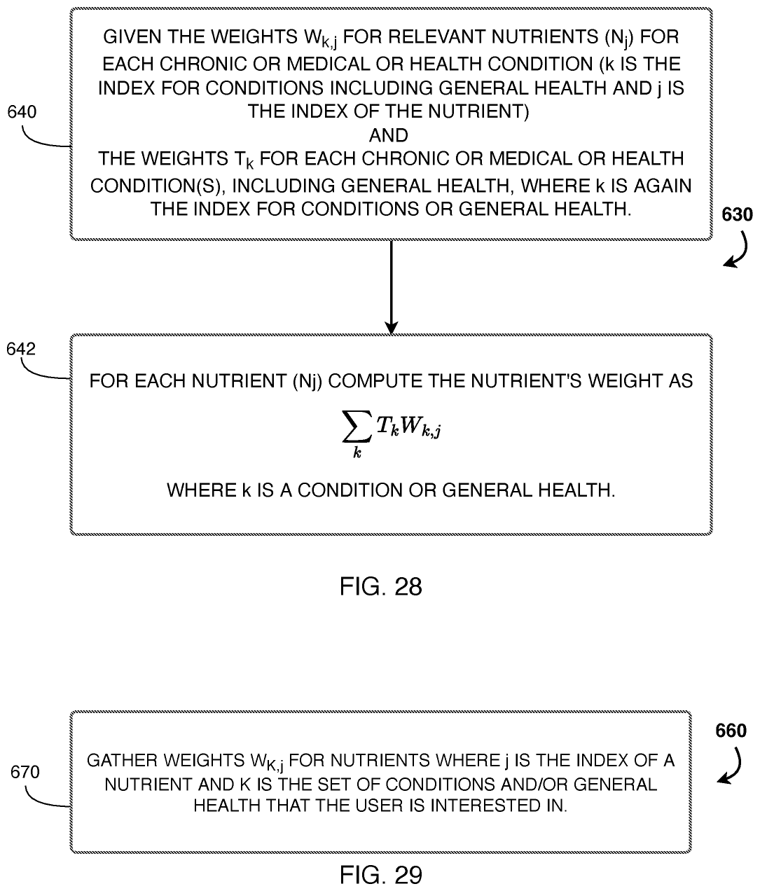

The flowchart 300 in FIG. 24 illustrates, in details, the various steps of at least one embodiment of the methods and systems specified in this disclosure. The flowchart further continues into 400 in FIG. 25 via the connector symbol A in step 334. In such embodiments, the step 422 is further illustrated in an expanded form in 500 in FIG. 26. Further, in such embodiments, step 526 may, as an example, be implemented using any one of the logic illustrated in 600, 630, or 660 in FIG. 27, FIG. 28, and FIG. 29 respectively. Those skilled in the art will readily understand that the logic illustrated in 600, 630, and 660 are not an exhaustive list and that several variations of the logic presented in 600, 630, and 660 may be employed to effect the calculations in step 526.

In flowchart 300, at step 310, the method receives a scoring request to score a recipe or food for a user. As indicated before, those skilled in the art will readily understand that this method can be easily extended to the case of handling one or more requests for scoring of recipes or foods for multiple users by iterating over all the users wherein in each iteration only one user is under consideration. In a similar way, those skilled in the art will readily understand that this method can be extended to scoring multiple recipes or foods by iterating over all the recipes wherein in each iteration only a single recipe or food is considered.

Details of the contents of a scoring request have been described earlier. At step 312, a check is done to ascertain whether all the relevant nutrients and their amounts are present in the scoring request. To create a list of names of relevant nutrients, a list of nutrient-preferences for the user is obtained from the data store(s) 116 or 128 or 134 or 148. The table 212 is stored, in relational databases, as a record in a table or a logical record across tables linked together using foreign keys and primary or unique keys or, in non-relational databases, as a document in a document-collection or a logical document spread across several document collections linked together using unique identifiers. An example of such a nutrient-preference record in a JSON (JavaScript Object Notation) format is depicted in the code snippet 230 in FIG. 12. Further additions are made to this list of names of relevant nutrients. These additions include names of nutrients for each of the chronic or medical condition(s) that the user may be interested in as well as for general health. In a fashion similar to the list of relevant nutrients, this list of the chronic or medical condition(s) the user may be interested in as well as for general health is stored in the data store(s) 116 or 128 or 134 or 148. An example of such as list 234 is shown FIG. 13A. The condition(s) that the user may be interested in may be specified in the scoring request. If the condition(s) are not specified in the scoring request, they can be obtained from the data store(s). An example of such data is shown in the code snippet 234 in FIG. 13A.

Once the list of relevant nutrients has been obtained for a user, the check in step 312 checks for the presence of all such nutrients and amounts of the nutrients in the scoring request. If not all the relevant nutrients are provided in the scoring request, a second check in step 314 may be done to see if the ingredients in the recipe or food may be used to obtain, for an amount of the recipe or food specified in the scoring request, the nutrients in the recipe or food.

If the ingredients are not specified in the scoring request, then a check in step 318 may be made to see if a unique identifier for a recipe or food has been provided in the scoring request. If no such unique identifier for a recipe or food has been provided in the scoring request, then since the data isn't sufficient to calculate the score for the recipe or food for the user in context of the condition that the user may be interested in, the scoring request is rejected in step 320 and the method stops in step 322. If, however, a unique identifier for a recipe or food has been provided in the scoring request, then in step 326 the details for the recipe or food with the unique identifier are obtained from the data store(s) 116 or 128 or 134 or 148 and the processing for the request continues to step 412. Examples of such details of the recipes or foods include names of nutrients and their amounts for 1 serving of the recipe or food as well as for 100 grams (and 100 ml as well if the recipe or food is better described in volume) of the recipe or food. The connector symbol A in step 334 in the flowcharts 300 and 400 is included to indicate the connection of the logic across the two figures FIG. 24 and FIG. 25.

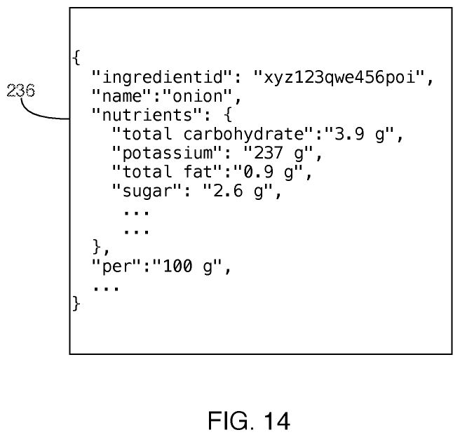

Going back to the check in step 314, while we looked at the logic of the method when the answer to the check in step 314 was a “NO”, here we will describe the logic of the method when the answer to the check in step 314 is a “YES”. In this case, when the check in step 314 identifies that one or more ingredients for the recipe or food and the amount of each respective ingredient is specified in the scoring request, the logic of the method continues to step 316. If the yield of the recipe or food is not specified in the scoring request, the yield is assumed to be 1 serving. The yield could be specified in terms of servings or units of weight (such as grams) or units of volume (such as 100 ml.) The yield of the recipe or food in weight (units of grams) or volume (units of ml) can be calculated from the ingredients. In step 316, the method goes over each of the ingredients and its weight in the request and, for each ingredient, the method gets the amount in units of weight (grams, milligrams etc.) for all the relevant nutrients from the data store(s) 116 or 128 or 134 or 148. An example record of an ingredient in the data store(s) in JSON format is illustrated in 236 in FIG. 14. The logic of the method then continues to step 324. In step 334, the method adds up the weights (grams, milligrams etc.) of each relevant nutrient across all the ingredients in the request. This results in a list of all relevant nutrients along with their weight (grams, milligrams etc.) for the recipe or food in the request. The logic in the method then continues to step 328.

Going back to the check in step 312, while we looked at the logic of the method when the answer to the check in step 312 was a “NO”, when the answer to the check in step 312 is a “YES” the method continues to step 328.

In the check in step 328, the method tests whether the scoring request asks to do the scoring for a particular serving size. If the answer is “NO”, then the method assumes, in step 330, the scoring request is asking to score the recipe or food at 1 serving. The logic then moves on to step 332. If the answer to the check in step 328 is “YES”, the logic moves directly to step 332 with the knowledge of the number of servings of the recipe or food in the scoring request. Those skilled in the art will note that in various embodiments the scoring request may request the score for a recipe or food for an amount measured not in serving size but in a measure of volume or weight. For example, the scoring request may ask the method to score, for a user, a recipe or food for, say, 135 grams or for, say, 200 ml.

In step 332, the method uses the serving size to change nutrient amounts for consideration in the following manner. To change the amounts for nutrients to correspond to the requested serving size, the method normalizes the amounts for nutrients for the amount of the recipe or food requested. For example, if the ingredients are specified for 5 servings and the scoring request is made for scoring of ⅔ serving, then the nutrient amounts are divided by 5 and then multiplied by ⅔.

In the check in step 412, the method checks for the presence of a unique user identifier in the scoring request. Such a unique user identifier is referred to in future as userid. If a userid is not in the scoring request, then the method will perform a check in step 414 to see if the scoring request includes a user's list of chronic or health or medical condition(s). If the scoring request does not have any chronic or health or medical condition(s) specified, then the method assumes that “general health” is the only condition that the user may be interested in. The method moves on to step 418.

At the check in step 414, if the scoring request includes one or more chronic or health or medical condition(s), i.e., the answer to the check in step 414 is a “YES”, then in step 418 the method obtains all the nutrients to consider for each chronic or health or medical condition(s) from table 218 for the purpose of scoring. The processing in the method then moves on to step 422.

At the check in step 414, if the scoring request does not include any chronic or health or medical condition(s); i.e., the answer to the check in step 414 is a “NO”, then in step 420 the method assumes that the request was for scoring of the recipe or food for “general health” and obtains all the nutrients to consider for “general health” from table 218 for the purpose of scoring. The processing in the method then moves on to step 422.

Going back to the check in step 412, if the scoring request includes a userid, then the answer to the check in step 412 is “YES” and the processing in the method moves to step 416. In step 416 the method gathers from table 214 in FIG. 8 the chronic or health or medical condition(s) that the user associated with userid may be interested in. In step 416 the method also gathers from table 212 any nutrient-preferences for the user associated with userid. The processing in the method then moves to step 422. After the scoring of the recipe has been completed in step 422 the method stops in step 424. Step 422 is expanded in the example flowchart 500 in FIG. 26.

While most of the steps in the example flowchart 500 are self-explanatory, some brief notes are provided below for a better understanding of the method.

Step 510 indicates that we begin with the knowledge of the amount Aⱼ of nutrient Nⱼ (where j is an index variable that ranges over all the know nutrients or a subset thereof) in one unit, wherein a unit may be a certain number (e.g. ½ or 1½or 2¾ etc.) of servings of the recipe or food, or a certain amount of weight (e.g., 80 grams or 12 ounces etc.), or a certain amount of volume (e.g., 50 ml etc.) of the recipe or food. At this step we also have the knowledge of the various chronic or health or medical condition(s) Cₖ (where k is an index variable that ranges over all chronic health and medical condition(s), including ‘general health’) including ‘general health’ that the target user may be interested in. At this step we also have the target user's nutrient-preferences Lₘ (where m is an index variable that ranges over all nutrients that the user has explicitly set).

At step 512, the method calculates the daily Dⱼ allowance of each nutrient Nⱼ for the target user. To calculate this daily allowance in this step, the method iterates over all the various chronic or health or medical condition(s) (including ‘general health’) the user may be interested in and collects the limits for each nutrient. If a nutrient occurs in multiple chronic or health or medical condition(s) then the method picks the more conservative value limit among all occurrences of that nutrient's limits. For example, if the nutrient total carbohydrates is a nutrient of consideration for the chronic or health or medical condition(s) PCOS and Type 2 diabetes, and limits for total carbohydrates is a maximum of 50% of daily calorie needs for PCOS and a maximum of 45% of daily calorie needs for Type 2 diabetes for the target user, then, both the limits being a maximum limits, the more conservative figure is a maximum of 45% of daily calorie needs. In cases where the limits for nutrients are contradictory, i.e., one or more of the chronic or health or medical condition(s) have maximum limits while one or more chronic or health or medical condition(s) have minimum limits, the limit corresponding to the most serious chronic or health or medical condition(s) is considered and the rest of the limits are ignored. If, however, the user has provided limits on certain nutrients that they would prefer to maintain, then those limits are considered for those nutrients and the limits for those nutrients obtained based on their chronic or health or medical condition(s) that they may be interested in are ignored.

At step 514, the method calculates for each nutrient Nⱼ, the percentage Pⱼ that the amount Aⱼ of the nutrient represents of the daily nutrient allowance Dⱼ. This is done simply by calculating the ratio Aⱼ/Dⱼ and converting that into a percentage to get Pⱼ.

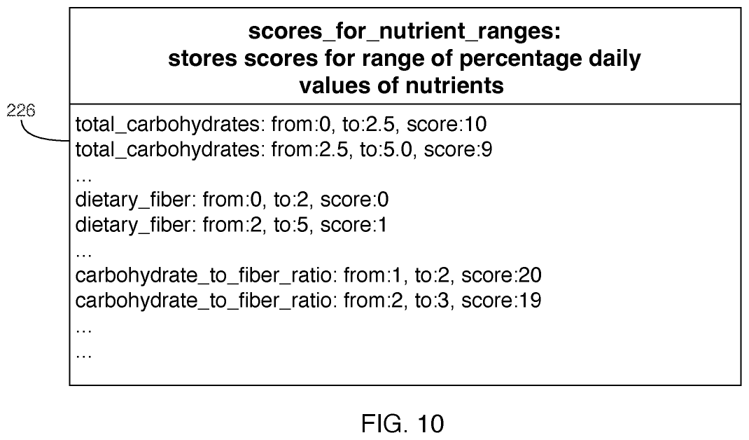

At step 516, the method calculates the score Sⱼ for each nutrient Nⱼ using the percentage Pⱼ that Aⱼ represents of the daily allowance Dⱼ. This is done utilizing the table 226 in FIG. 10. The table has a list of all known nutrients. For each nutrient the table 226 has the range 0% to 1000% or some higher number, divided into mutually exclusive but completely exhaustive (i.e., non-overlapping but completely covering the range from 0% to 1000% or some higher number) subsets where each subset or sub-range corresponds to a score. For example, as illustrated in the example table 226, if the P_{dietary}_{_{_}}_{fiber} for the nutrient dietary fiber for, say, ½ a serving of a certain recipe or food is 2.8% of the daily need for a certain target user, then the score S_{dietary}_{_{_}}_{fiber} for this nutrient, dietary fiber, for that amount of the recipe or food for the user is 1.

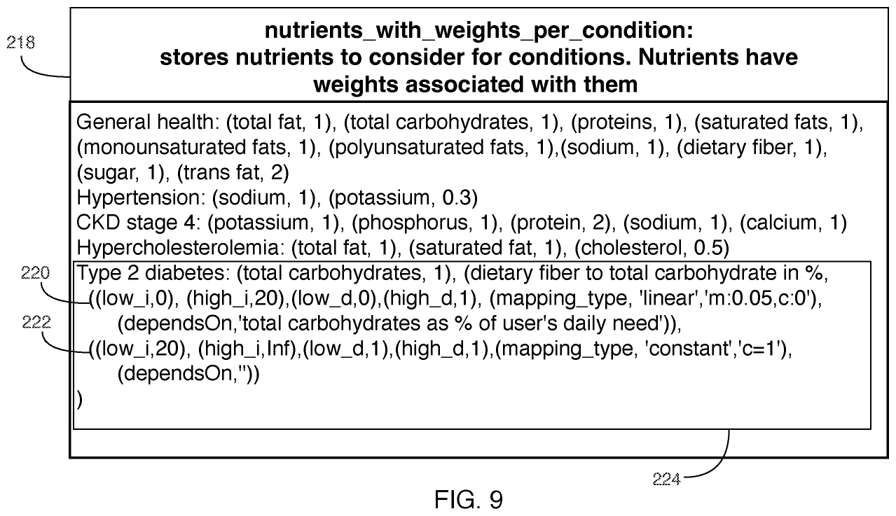

At step 518, the method iterates over each chronic or health or medical condition(s) Cₖ the target user may be interested in. Such conditions include ‘general health’. In each iteration, one and only one of the chronic or health or medical condition(s), including ‘general health’, Cₖ is considered. In each iteration, the method gathers the names of nutrients that are relevant for the condition Cₖ under consideration for the iteration and the weights W_{k,j} (wherein k is an index variable that ranges over all chronic health and medical condition(s), including ‘general health’ and j is an index variable that ranges over all the know nutrients or a subset thereof) of these nutrients for the condition Cₖ. The weights W_{k,j} are used to emphasize that certain nutrients are more important than others for the chronic or health or medical condition(s) under consideration. The method gathers these W_{k,j} from the data store(s) 116 or 128 or 134 or 148 where the weights are kept in a table similar to the one illustrated in an example table 218 in FIG. 9. These weights W_{k,j} may have either a fixed value or a non-fixed (i.e., varying) value. In FIG. 9, each condition may be associated with one or more nutrient preferences. Each such nutrient-preference may be associated with a fixed value of weight or a specification of a varying value of weight.

An example of fixed value of the weight W_{k,j} is shown in the table 218 for the entry corresponding to the condition Cₖ of ‘Hypercholesterolemia’ and nutrient Nⱼ of ‘total fat’ where the weight for the nutrient has a fixed value of 1.

An example of varying value of the weight W_{k,j} is also shown in an example record 224 in the table 218. The example record 224 corresponding to the condition Cₖ of ‘Type 2 diabetes’ and nutrient Nⱼ of ‘dietary fiber to total carbohydrate in %’ illustrates the example. The weight illustrated in the example is for the computed nutrient ‘dietary fiber to total carbohydrate in %’ which is obtained by dividing the amount of dietary fiber in a given amount of a food by the amount of total carbohydrate in that food and expressed as a percentage. A ‘computed nutrient’ is defined as an entity whose value is obtained by way of performing mathematical operations on one or more other dietary nutrients or computed nutrients. In this example of varying value of the weight of a nutrient, the weight has two regimes. A regime may be defined as a range of values of an ‘independent nutrient.’ A regime may also, at times, be defined as a range of values of weights of ‘dependent nutrients.’ In this case the first definition is more pertinent. In this context of varying value of weights, dependent nutrients and independent nutrients are defined in the following way. A nutrient is called a ‘dependent nutrient’ if its weight depends on the amount of another nutrient in a given food. A nutrient P is called an ‘independent nutrient’ if the weight of another nutrient Q depends on this first nutrient P. Here, ‘total carbohydrates as % of user's daily need’ is an independent nutrient and ‘dietary fiber to total carbohydrate in %’ is a dependent nutrient.

The example record 224 comprises two regimes. The first regime is illustrated by the section of the record indicated by 220 and the second regime is illustrated by the section of the record indicated by 222. In the first regime, as indicated by the value for the field ‘dependsOn’ in section 220 of the record 224, the value of the weight for the dependent nutrient ‘dietary fiber to total carbohydrate in %’ depends on the value for the independent nutrient ‘total carbohydrates as % of user's daily need’. Also, the value of the independent nutrient varies from 0 as indicated by the entry for ‘low_i’ (‘low_i’ is a short form for ‘lower limit for the value for the independent nutrient’) to a value of 20 as indicated by the entry for ‘high_i’ (‘high_i’ is a short form for ‘upper limit for the value for the independent nutrient’) and correspondingly, the value of the weight of the dependent nutrient varies from a value of 0 as indicated by the entry for ‘low_d’ (‘low_d’ is a short form for ‘lower limit for the value of the weight for the dependent nutrient’) to a value of 1 as indicated by the entry for ‘high_d’ (‘high_d’ is a short form for ‘upper limit for the value of the weight for the dependent nutrient’). The nature of relationship between the value for the independent nutrient and the weight for the dependent nutrient is illustrated by the specification in the mapping_type sub-record: “(mapping_type, ‘linear’, ‘m:0.05, c:0’)”. The second entry of this sub-record indicates that the relationship is ‘linear’. This linear relationship is further specified by the values of the parameters as illustrated by the third entry for the same mapping_type sub-record which states that the parameters for the ‘linear’ relationship are ‘m:0.05, c:0’. The ‘m’ and ‘c’ refer to the generally used linear relationship presented by the equation Y=m*X+c, where X is the independent variable and Y is the dependent variable. In this method, in the context of varying value of weights of nutrients for scoring of foods for certain chronic or health or medical condition(s), when the relationship being indicated via the mapping_type sub-record is linear, the weight for the dependent nutrient is represented by Y and the value for the independent nutrient is represented by X in the above equation. In summary, as an illustration of the varying value of weight of a nutrient, the sub-record 220 is saying that, for assessing the score of a food for the condition ‘Type 2 diabetes’, in order to obtain the weight for the factor corresponding to the computed nutrient ‘dietary fiber to total carbohydrate in %’, if the value for the independent nutrient ‘total carbohydrates as % of user's daily need’ in the food is between 0% to 20% (of the user's total daily calorie needs,) the method uses the following formula ‘weight of ‘nutrient dietary fiber to total carbohydrate in percent’=0.05*total carbohydrates as % of user's daily need’+0. For further illustration purposes, if the food contains total carbohydrates amounting to 10% of the user's daily needs of total carbohydrates, then, when calculating the score of the food for the user for the condition ‘Type 2 diabetes’, if the user has expressed a need for getting a score for this condition, the weight assigned to the computed nutrient ‘dietary fiber to total carbohydrate in %’ may be calculated as 0.05*10+0 which amounts to 0.5.

The second regime in the example record 224 is illustrated by section 222 of the record. This second regime applies for the range of the value for the independent nutrient from 20 as indicated by ‘low_i’ to ‘Inf’ as indicated by ‘high_i’ in the section 222 of the record 224. The value ‘Inf’ merely indicates that the value has no limit though in real life there may be a reasonable limit. Furthermore, as indicated in the same section, the value for the weight of the dependent nutrient varies from a value of 1 indicated by ‘low_d’ to a value of 1 indicated by ‘low_d’ and the nature of the relationship between the value for independent nutrient and the weight of the dependent nutrient is ‘constant’ as indicated in the same section 222 of record 224 by the sub-record for mapping_type: (mapping_type, ‘constant’,‘c=1’). In summary, the section 222 indicates that for the range of values for the independent nutrient above 20, the weight of the dependent nutrient stays constant at 1.

While in the sections 220 and 222 of the example record 224, which are for illustration purposes, we have shown examples of a ‘linear’ and a ‘constant’ relationship, the method may incorporate other types of relationships including but not limited to various non-linear relationships including polynomial or exponential or logarithmic or logistic relationships, or combinations thereof.

At step 520, the method again iterates over each chronic or health or medical condition(s), including ‘general health’, Cₖ that the target user may be interested in. In each iteration, one and only one chronic or health or medical condition(s), including ‘general health’, Cₖ is considered. In each iteration, the method computes the score for the recipe or food for the user by calculating the weighted average of the scores of all the nutrients that are relevant for the chronic or health or medical condition(s) Cₖ that the user may be interested in. A weighted average is a fraction where the numerator is the weighted sum of the scores of the nutrients and the denominator is the sum of the weights of the nutrients considered for the scoring. For example, let's assume a user is interested in a chronic or health or medical condition Cₖ. Let's further assume that Nⱼ₁ being the nutrient ‘total carbohydrates’, the value of weight W_{k,j1} is 1. Let's also assume that Nⱼ₂ being sodium, the value of weight W_{k,j2} is 0.5. Additionally, for this example, if the recipe or food, at the requested amount, say ½ of a serving, has ‘total carbohydrates’ at 5% of daily need of the user indicating a score Sⱼ₁ of, say, 8, and has sodium at 8% of daily need of the user indicating a score S_{J2} of, say, 6, then the score of the ½ of a serving of the recipe or food for the user for condition k is (1*8+0.5*6)/(1.5). Here the numerator is the weighted sum of the scores for the relevant nutrients considered for the scoring and the denominator is the sum of weights of the same relevant nutrients.

In some embodiments, to score a nutrient based on the percentage of daily need of a user for the nutrient, a lookup may be done against a table similar to the sample of an example table shown in 226. To generate the ranges in such a table, in some embodiments, ‘anchor’ limits to the percentage of daily need for a nutrient may first need to be defined. For example, for carbohydrates, a certain amount of a recipe or food which has carbohydrate amount less than 5% of daily need of a target user may be deemed to be low in carbohydrates. Here the 5% of daily need is an ‘anchor’ limit. Also, a certain amount of another recipe or food which has carbohydrate amount higher than 20% of the daily need of a user may be deemed to be high in carbohydrates. Here the 20% of daily need is an ‘anchor’ limit. With the aforementioned two ‘anchor’ limits defined, the method can use these values: 5% and 20%, as anchors for setting up sub-ranges for scoring of this nutrient, i.e., carbohydrates. The method, in some embodiments, may be configured to divide the range between the anchor points into a certain number of sub-ranges of equal or varying sizes and assign each sub-range a score. For example, the method may be configured with the following six sub-ranges between 5% and 20% for carbohydrates: [5%, 7.5%) has a score of 8, [7.5%, 10%) has a score of 7, [10%, 12.5%) has a score of 6, [12.5%, 15%) has a score of 5, [15%, 17.5%) has a score of 4, [17.5%, 20%) has a score of 3.

In the example sub-ranges defined for carbohydrates above, the terminology [percentage1, percentage2) indicates a sub-range where a value v is in the sub-range if it satisfies the condition in 276 in FIG. 23.

Additional sub-ranges may be defined to cover any other non-zero percentage of daily need values of the nutrient. In this example, the sub-ranges to cover may be [0%, 5%) and any percentage values over 20%. The sub-ranges may again be divided into smaller sub-ranges with scores assigned to each sub-range. In this example, the subrange [0%, 5%) may be further divided in the following manner: [0%, 2.5%) has a score of 10, [2.5%, 5%) has a score of 9.

Additionally, in this example, for percentage values over 20%, the sub-ranges may be defined as following: [20%, 22.5%) has a score of 2, [22.5%, 25%) has a score of 1, [25%, 30%) has a score of 0, [30%, 35%) has a score of −1, [35%, 40%) has a score of −2, [40%, 50%) has a score of −3, [50%, 60%) has a score of −4 etc. with the subrange increasing by 10 and the score decreasing by 1 to more negative values.

For nutrients whose lower consumption may be desired, the scores associated with the sub-ranges may follow a pattern similar to that shown with the aforementioned example of carbohydrate. Here, the scores for sub-ranges fall as the percentage values of the sub-ranges increase, the score being the same within the range, but falling as the percentage values increase across the sub-ranges. For nutrients whose higher consumption may be desired, e.g., dietary fiber or in some cases proteins or in yet some other cases potassium, the scores associated with the sub-ranges may follow a pattern opposite to that shown with the aforementioned example of carbohydrate. In such cases, the scores for sub-ranges may rise across the sub-ranges as the percentage values of the sub-ranges increase. An example of such a distribution of scores for subranges for, say, dietary fiber may be the following: [0%, 2.5%) has a score of −1, [2.5%, 5%) has a score of 0, [5%, 7.5%) has a score of −1, [7.5%, 10%) has a score of 2, [10%, 12.5%) has a score of 3, [12.5%, 15%) has a score of 4, [15%, 17.5%) has a score of 5, [17.5%, 20%) has a score of 6, [20%, 22.5%) has a score of 7, [22.5%, 25%) has a score of 8, [25%, 30%) has a score of 9, [30%, 40%) has a score of 10, [40%, 50%) has a score of 11, etc., with the subrange increasing by 10 and the score increasing by 1 to more positive values.

At the check in step 522, the method checks if the system is configured for the user to report the worst score of the various chronic or health or medical condition(s) that the target user may be interested in as the overall score of the recipe or food for the target user. If the answer is a “YES”, the processing moves to step 524. If, however, the answer to the check in step 522 is a “NO”, the logic of the method moves to step 526. In various embodiments, the logic in step 526, may be implemented in several ways or mechanisms, a subset of which is presented in 600, 630 and 660 in FIG. 27, FIG. 28 and FIG. 29 respectively. While many of the steps in 600, 630 and 660 are self-explanatory, a brief description of some of the steps has been provided below. The various mechanisms provide weights for each relevant nutrient. These weights are used to calculate the overall score of the recipe or food for the target user. Once the weights are obtained, in step 526, a weighted average is computed over the scores of the relevant nutrients to obtain the overall score of the recipe or food for the target user. Those skilled in the art will note that a standard definition of weighted average is being used in the calculations here. As an illustration, for an example scenario, let's assume that for a given scoring request for a recipe or food there are three relevant nutrients: Nₐ, N_{b} and N_{c} which have weights of 1, 4 and 4, respectively, and have scores of Sₐ, S_{b} and S_{c}, respectively. For this example scenario, the weighted average score, which is the overall score of the recipe or food, may be calculated as: (1*Sₐ+4*S_{b}+4*S_{c})/(1+4+4).

At step 528, once the overall score O as well as the condition specific scores Kₖ (where k is an index variable that ranges over all chronic health and medical condition(s), including ‘general health’) for any zero or more chronic or health or medical condition(s) has been computed and passed to this step from the previous steps 524 or 526, the scores may be converted into color codes or letter grades for convenient presentation to the user, for example, on the screen of a device or sent as a part of response to an API scoring request from a requesting entity. The scores may be presented along with the colour codes or letter grades. The scores may also be presented just by themselves. The mapping from scores to color codes or letter grades may be done using a one or more table(s) stored in the data store(s) 116 or 128 or 134 or 148. An example record 240 for mapping of overall score O to a color code and letter grade from a table that illustrates such a mapping is shown in FIG. 16. An example record 240 for mapping of overall score O to a color code and letter grade illustrates such a mapping is shown in FIG. 16. An example record 242 for mapping of scores for various chronic or health or medical condition(s) Kₖ to a color code and letter grade illustrates such a mapping is shown in FIG. 17. Example records 240 and 242 and similar such records may be stored in the data store(s) 116 or 128 or 134 or 148.

In various embodiments, in the alternative mechanism 600, at step 610, the method has all the weights W_{k,j} for each relevant nutrient Nⱼ for each of the various chronic or health or medical condition(s) Kₖ.

In various embodiments, in the alternative mechanism 600, at step 612, the method iterates over all the relevant nutrients for the conditions that the target user may be interested in. In each such iteration, for a given nutrient Nⱼ, the method adds up the weights for that nutrient across all the various chronic or health or medical condition(s) the target user may be interested in. The resultant sum is the weight of the nutrient Nⱼ for calculating the overall score O of a recipe or food for the target user in step 526.

In various embodiments, in the alternative mechanism 630, at step 640, the method has all the weights W_{k,j} and for each relevant nutrient Nⱼ for each of the various chronic or health or medical condition(s) Kₖ. Additionally, the method also has the weights Tₖ (where k is an index variable that ranges over all chronic health and medical condition(s), including ‘general health’) for each of the various chronic or health or medical condition(s) the target user may be interested in.

In various embodiments, in the alternative mechanism 630, at step 642, the method iterates over all the relevant nutrients for the various chronic or health or medical condition(s) that the target user may be interested in. In each such iteration, for a given nutrient Nⱼ, the method calculates a weighted sum of the weights of the nutrient Nⱼ across all the various chronic or health or medical condition(s) the target user may be interested in. The resultant weighted sum is the weight of the nutrient N, calculating the overall score O of a recipe or food for the target user in step 526. As an alternative to the weighted sum, the method may choose to apply another function to over the set of weights, for example, the function may be a ‘maximum’ function, which would choose the largest weight for the nutrient N, across the various conditions, or the function may be an ‘mean’ function, which would calculate an average of the weights of the nutrient Nⱼ across the various chronic or health or medical condition(s). Some other alternative functions may be median, minimum, etc.

As an illustration of the mechanism in 642, an example is provided in the following. Let's assume that the scoring request is for a target user who is interested in two conditions C₁ and C₂. Let's assume that the two conditions C₁ and C₂ have weights T₁ and T₂, respectively. Let's assume that T₁ has a value of 1 and T₂ has a value of 2.Let's assume that nutrients Nₐ and N_{b} are relevant for Ci and each has a weight of 1 and 2 respectively. Let's also assume that nutrients N_{b} and N_{c} are relevant for C₂ and each has a weight of 1 and 2 respectively. Let's also assume that the nutrients Nₐ, N_{b} and N_{c} have been scored Sₐ, S_{b} and S_{c} for the recipe of food in the scoring request for the target user who is interested in conditions C₁ and C₂. Given these assumptions in this illustrated example, the weights of the nutrients for the target user for the recipe of food in the scoring request is as follows: (a) for nutrient Nₐ the weight is T₁*1 which amounts to 1*1 which in turn amounts to 1, (b) for nutrient N_{b} the weight is T₁*2+T₂*1 which amounts to 1*2+2*1 which in turn amounts to 4, and finally, (c) for nutrient N_{c} the weight is T₂*2 which amounts to 2*2 which in turn amounts to 4.

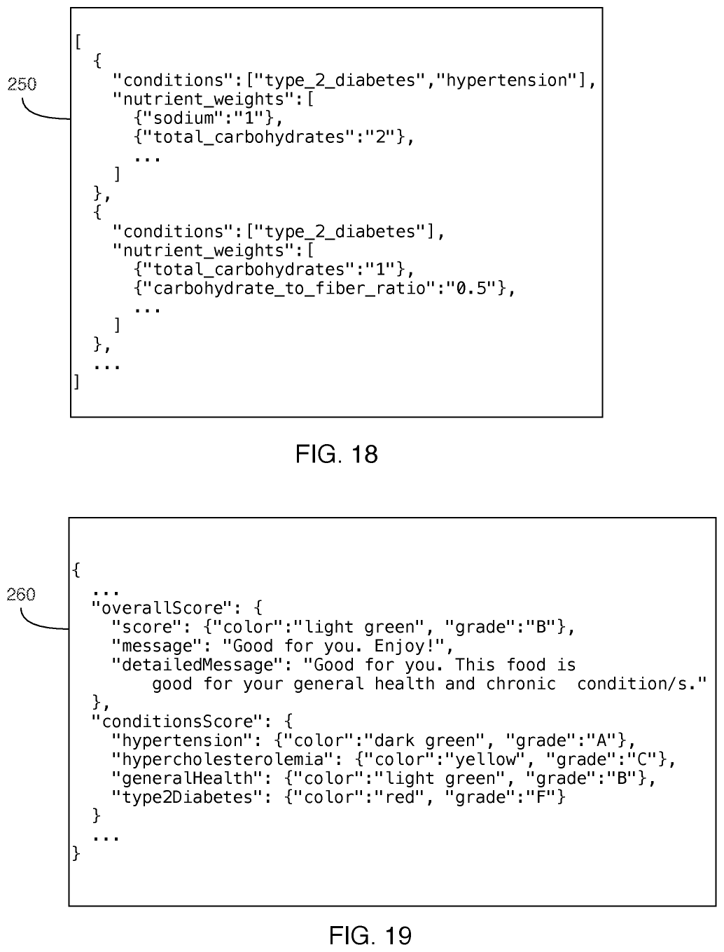

In various embodiments, in the alternative 660, at step 670, the method maintains a list of weights for all known relevant nutrients for each combination of various chronic or health or medical condition(s). The list of weights are maintained in the data store(s) 116 or 128 or 134 or 148. A code snippet 250 of an example of records storing such a list is illustrated in FIG. 18. For the target user a record corresponding to the set K of various chronic or health or medical condition(s) that the target user is interested in is obtained. From this record, weights W_{K,j} for the various known relevant nutrients Nⱼ are obtained. These weights may be used for calculating the overall score O of a recipe or food for the target user in step 526.

The user interface 700 in FIG. 30 is an example user interface for displaying to the user the scores for a selected amount of a recipe or food for a user who is interested in a set of chronic or health or medical condition(s). In various embodiments, the user may be able to select or change the amount of the recipe or food (selection or change interface indicated in 718, 740, and 760) which may result in the methods and systems computing the score of the recipe or food and presenting the appropriate color code (714, 736, and 756) and letter grade (712, 734, and 754) for the recipe or food for the various chronic or health or medical condition(s) (716, 738, and 758) that the target user is interested in.

In user interface 700, for the selected amount of ½ of a serving of the recipe or food, by the application of an embodiment of the methods and systems outlined above, for the user, the scores for a set of chronic or health or medical condition(s) as calculated are shown. In user interfaces 730 and 750 in FIG. 31 and FIG. 32, respectively, the selected amount has been changed to 1 and 1½ servings, respectively, for the same recipe or food as that in user interface 700. As a result of this increase in the amount of the recipe or food, the scores calculated by the methods and systems outlined above, for the same user and for the same set of chronic or health or medical condition(s) as that for user interface 700, change to account for the change in the amount of the recipe or food.

The code snippet 260 in FIG. 19 illustrates a sample of an example of an API response for a scoring request for scoring of a certain amount of a recipe or food for a user who may be interested in a set of chronic or health or medical condition(s). The set of chronic or health or medical condition(s) for the sample response illustrated in 260 includes hypertension, hypercholesterolemia, type 2 diabetes and general health. The example response 260 illustrates that the API response may include an overall score comprising a color code and a letter grade as well as scores for each condition again comprising a color code and a letter grade.

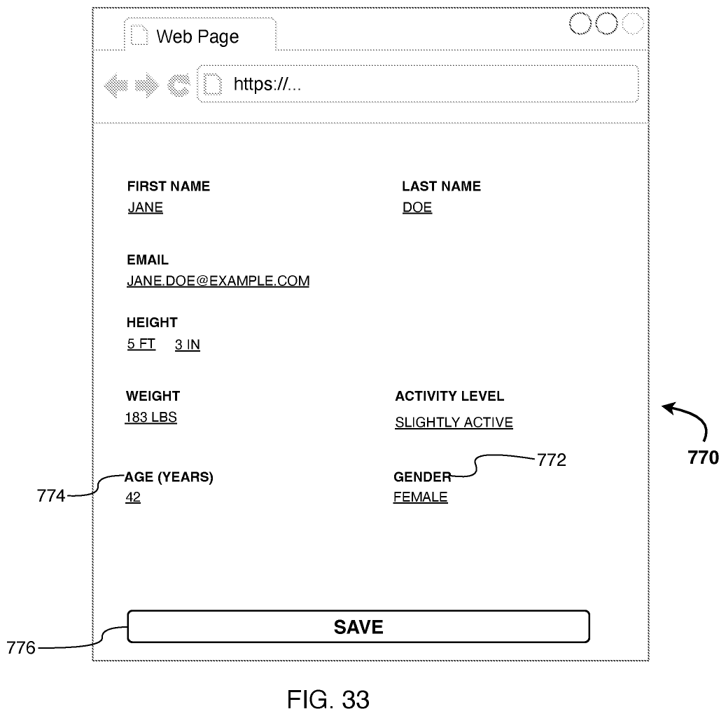

The user interface 770 in FIG. 33 illustrates an example user interface using which a target user may set their medical and biological information including but not limited to weight 774, activity-level 772, age 776 etc. Such medical and biological information is used to estimate the target user's daily calorie needs using such methods as the Harris-Benedict method, Mifflin St Jeor method etc.

The user interface 780 in FIG. 34 illustrates an example user interface using which a target user may set their preferences for limits on consumption of nutrients per unit of time, usually a day. A user may be able to set preferences on limits for any and all nutrients known in the system. The list of nutrients illustrated in 780 is a sample of nutrients known in the system. These preferences are stored in the data store(s) such as 116 or 128 or 134 or 148. An example of such a nutrient-preference record may be stored in a JSON (JavaScript Object Notation) format is depicted in the code snippet 230 in FIG. 12. The nutrient limit-preferences may be a maximum limit or a minimum limit, where a maximum limit indicates that the recipe or food scoring should assume that the target user prefers to have an upper bound on the consumption of the nutrient for which the maximum limit has been set. Correspondingly, a minimum limit indicates that the recipe or food scoring should assume that the target user prefers to have a lower bound on the consumption of the nutrient for which the minimum limit has been set. Additionally, each limit could be either a limit on an absolute amount (in units of weight, e.g., grams or mcg. or volume, i.e., ml or fl oz.) of the nutrient, whether minimum or maximum, or a limit on an amount that corresponds to a percentage of the daily calorie needs of the target user, e.g., 10% of daily calorie need of the target user. An example of a limit on an absolute amount is shown in the limit for Cholesterol (788) in user interface 780. An example of a limit on an amount that corresponds to a percentage of the daily calorie needs of the target user is shown for Carbohydrate (782) in user interface 780. In the example, for the limit on carbohydrate, the user is shown the absolute amount (786) based on the percentage (784) of the user's daily calorie needs set by the user for the user's knowledge and understanding. The example code snippet 230 in FIG. 12 is an example of a record from the table 212 in FIG. 7.

In some embodiments, the message module(s) 164 may be configured to construct or create at least one message based, at least, on at least one of: (a) the overall score of a recipe or food for a target user, or (b) one or more of the condition scores Kₖ of the recipe or food, or (c) the amounts or scores for one or more nutrients that the target user may be interested in, or (d) the amounts or scores for one or more nutrients, selection of such nutrients being based on the one or more chronic or medical condition(s) that the target user may be interested in, or (e) any combination of (a), (b), (c) and (d). The output of the message module(s) 164 may be used to display feedback to the user similar to the section 710, 732, and 752 in the example user interfaces. Those skilled in the art will note that, as noted elsewhere, in various embodiments, the messages may be a part of the response sent by the API server(s) or module(s) 178.

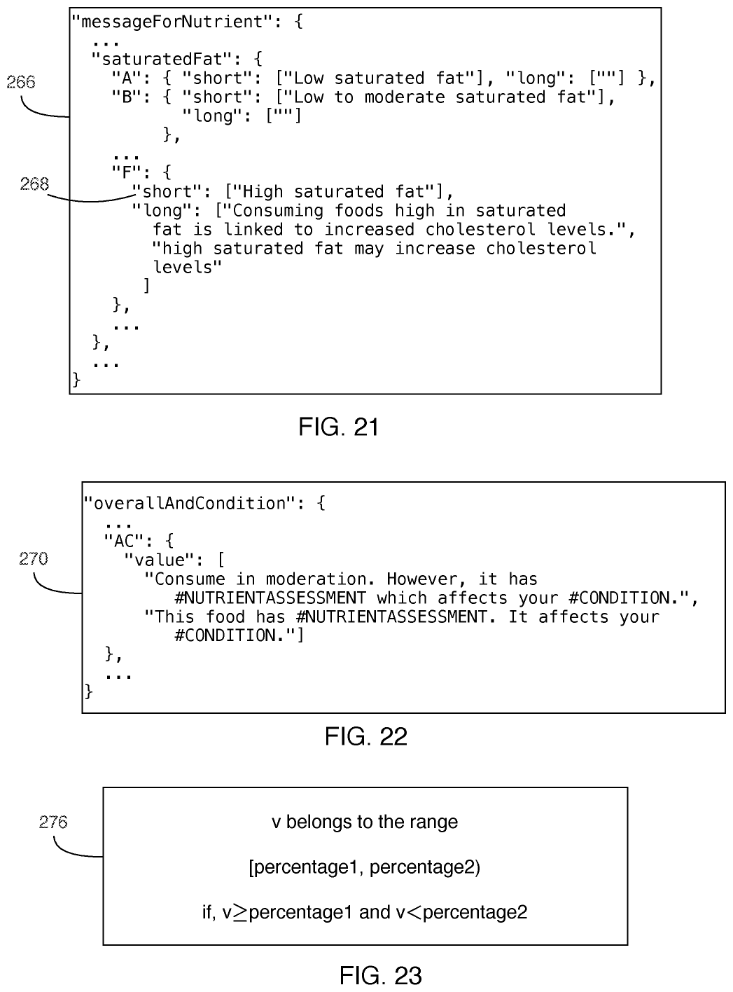

The code snippet 266 in FIG. 21 illustrates an example configuration which may be used to generate messages for certain nutrients based, at least, on the score for the nutrient for a recipe or food at a certain amount or serving for a target user. Similarly, the code snippet 270 in FIG. 22 illustrates an example configuration which may be used to generate messages for the overall score of a recipe or food for the target user. Those skilled in the art will note that while in the code snippet 270 in FIG. 22, the value for the key “value” is an array that has, for this illustration only, two entries, any one or more of which may be selected to construct the message, an actual implementation may have, for this configuration, any number of entries in the array for the key “value” in the code snippet 270 in FIG. 22. Similarly, those skilled in the art will note that in code snippet 266 in FIG. 21, the values corresponding to the keys “short” and “long” may contain more than one entry, of which any one or more may be chosen to construct the message. After the scoring module(s) 170 has/have computed the score for the recipe or food and for the nutrients that constitute the recipe or food for the user for one or more condition(s) or ‘general health’ or a combination thereof, in order to generate the appropriate message, the coordination module(s) 176 may invoke the message module(s) 164. In some embodiments, the message generated becomes part of the score for the recipe or food.

As an example of messages for nutrients, if the saturated fat (which is a nutrient) content of a recipe is scored an ‘F’ by the method, then the message module(s) 164 may, for illustration purpose in this example, using the values as illustrated in 266, generate two messages: (a) ‘High saturated fat’ and (b) ‘Consuming foods high in saturated fat is linked to increased cholesterol levels’. In some embodiments, the score for the food in the illustrated example comprises “F,” “high saturated fat,” and “Consuming foods high in saturated fat is linked to increased cholesterol levels.”

As an example of messages for the overall score, if, for the overall score of the recipe or food for the target user, the letter grade is A and, say, the average letter grade for all the various condition(s) including ‘general health’ is C, then the method may pick up the message shown in the code snippet 270 of the example configuration. The term ‘#NUTRIENTASSESSMENT’ may, for example, be replaced by a comma separated list of the short messages 268 for all or a subset of the nutrient(s) that are relevant for all or any of the condition(s) that the user may be interested in. The subset may be chosen based on a mechanism configured in the system. Some examples of this mechanism may include the following: (1) if the overall score indicates that the recipe or food is bad for the target user, then pick the worse nutrients, or (2) pick those nutrients that have an extreme (bad or good) scores amongst all the relevant nutrients, or (3) pick only the nutrients for the most significant of the condition(s) where the significance may be determined based on any salient attributes of the nutrients etc., a combination of (1), (2) and (3). The term ‘#CONDITION’ may, for example, be replaced by a comma separated list of the following: (1) the condition(s) that the target user may be interested in, or (2) the conditions for which the nutrient(s) chosen for ‘#NUTRIENTASSESSMENT’ are significant as per the example table 218 etc.

All methods described herein can be performed in any suitable order unless otherwise indicated herein or otherwise clearly contradicted by context.

A recitation of ranges of values herein are merely intended to serve as a shorthand method of referring individually to each separate value falling within the range, unless otherwise indicated herein, and each separate value is incorporated into the specification as if it were individually recited herein. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a range of 1-3 refers to 1, 2, or 3 components. Similarly, a range of 1-5 refers to 1, 2, 3, 4, or 5 components, and so forth.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

Exemplary embodiments of the systems and methods are described above in detail. The systems and methods are not limited to the specific embodiments described herein, but rather, components of the systems and/or steps of the method may be utilized independently and separately from other components and/or steps described herein.

This written description uses examples to disclose the present embodiments, including the best mode, and also to enable any person skilled in the art to practice the present embodiments, including making and using any systems or performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

We claim:

1. A computer implemented method for scoring a food or recipe for its suitability to one or more medical conditions inputted by a user of an application program configured to carry out the method, comprising:
receiving, from a user computing device, a user-generated query for the food or recipe, wherein the food or recipe comprises one or more food components, wherein each respective food component comprises one or more nutrients;
retrieving the one or more nutrients for each respective food component and an amount of each respective nutrient;
determining a cumulative amount of each nutrient for the food or recipe;
determining a per serving amount of each nutrient for the food or recipe;
receiving, from the user computing device, the one or more medical conditions inputted by the user;
retrieving a default set of nutrient preferences for each respective medical condition, wherein the default set of nutrient preferences comprises a recommended minimum amount or maximum amount for one or more medical condition-specific nutrients;
receiving, from the user computing device, a confirmation or modification of the default set of nutrient preferences for each respective medical condition, wherein the modification is configured to override the default set of nutrient preferences for one or more nutrients;
determining, for each respective medical condition, a first score indicating the suitability of the food or recipe for the user; and
transmitting, to the display of the user computing device, the first score.

2. The computer implemented method of claim 1, wherein the receiving, from the user computing device, the one or more medical conditions inputted by the user comprises a plurality of medical conditions.

3. The computer implemented method of claim 2, wherein each of the plurality of medical conditions inputted by the user is assessed a first initial weight of relative importance by the application program.

4. The computer implemented method of claim 3, further comprising:
receiving, from the user computing device, a confirmation or modification of the assessed first initial weight of relative importance for each respective medical condition, wherein the modification is configured to override the first initial weight assessed by the application program.

5. The computer implemented method of claim 4, wherein the first initial weight of relative importance for each of the plurality of medical conditions inputted by the user is equal.

6. The computer implemented method of claim 5, wherein the receiving, from the user computing device, a confirmation or modification of the assessed first initial weight of relative importance for each respective medical condition is a modification; and
wherein the modification assesses a first user-specific weight of relative importance for each of the plurality of medical conditions and the respective first user-specific weights are not equal.

7. The computer implemented method of claim 2, further comprising:
determining, for all the one or more medical conditions, a second score indicating the suitability of the food or recipe for the user; and
transmitting, to the display of the user computing device, the second score.

8. The computer implemented method of claim 1, wherein the default set of nutrient preferences for at least one of the one or more respective medical conditions comprises a plurality of medical condition-specific nutrients, and
wherein each of the plurality of medical condition-specific nutrients is assessed a second initial weight of relative importance by the application program.

9. The computer implemented method of claim 8, wherein the receiving, from the user computing device, a confirmation or modification of the default set of nutrient preferences for each respective medical condition is a modification; and
wherein the modification assesses a second user-specific weight of relative importance for each of the plurality of medical condition-specific nutrients.

10. The computer implemented method of claim 1, further comprising:
receiving, from a user computing device, a modification of the number of servings for the food or recipe; and
determining, for each respective medical condition, the first score indicating the suitability of the food or recipe for the user based on the modified number of servings.

11. A computer program product comprising a non-transitory computer readable storage medium having program instructions embodied therewith, the program instructions executable by a computer processor to cause the computer processor to perform a method, comprising:
providing an application program for carrying out the method;
receiving, from a user computing device, a user-generated query for the food or recipe, wherein the food or recipe comprises one or more food components, wherein each respective food component comprises one or more nutrients;
retrieving the one or more nutrients for each respective food component and an amount of each respective nutrient;
determining a cumulative amount of each nutrient for the food or recipe;
determining a per serving amount of each nutrient for the food or recipe;
receiving, from the user computing device, the one or more medical conditions inputted by the user;
retrieving a default set of nutrient preferences for each respective medical condition, wherein the default set of nutrient preferences comprises a recommended minimum amount or maximum amount for one or more medical condition-specific nutrients;
receiving, from the user computing device, a confirmation or modification of the default set of nutrient preferences for each respective medical condition, wherein the modification is configured to override the default set of nutrient preferences for one or more nutrients;
determining, for each respective medical condition, a first score indicating the suitability of the food or recipe for the user; and
transmitting, to the display of the user computing device, the first score.

12. The computer program product of claim 10, wherein the receiving, from the user computing device, the one or more medical conditions inputted by the user comprises a plurality of medical conditions.

13. The computer program product of claim 12, wherein each of the plurality of medical conditions inputted by the user is assessed a first initial weight of relative importance by the application program.

14. The computer program product of claim 13, further comprising:
receiving, from the user computing device, a confirmation or modification of the assessed first initial weight of relative importance for each respective medical condition, wherein the modification is configured to override the first initial weight assessed by the application program.

15. The computer program product of claim 14, wherein the first initial weight of relative importance for each of the plurality of medical conditions inputted by the user is equal.

16. The computer program product of claim 15, wherein the receiving, from the user computing device, a confirmation or modification of the assessed first initial weight of relative importance for each respective medical condition is a modification; and
wherein the modification assesses a first user-specific weight of relative importance for each of the plurality of medical conditions and the respective first user-specific weights are not equal.

17. The computer program product of claim 12, further comprising:
determining, for all the one or more medical conditions, a second score indicating the suitability of the food or recipe for the user; and
transmitting, to the display of the user computing device, the second score.

18. The computer program product of claim 10, wherein the default set of nutrient preferences for at least one of the one or more respective medical conditions comprises a plurality of medical condition-specific nutrients, and
wherein each of the plurality of medical condition-specific nutrients is assessed a second initial weight of relative importance by the application program.

19. The computer program product of claim 18, wherein the receiving, from the user computing device, a confirmation or modification of the default set of nutrient preferences for each respective medical condition is a modification; and
wherein the modification assesses a second user-specific weight of relative importance for each of the plurality of medical condition-specific nutrients.

20. The computer program product of claim 10, further comprising:
receiving, from a user computing device, a modification of the number of servings for the food or recipe; and
determining, for each respective medical condition, the first score indicating the suitability of the food or recipe for the user based on the modified number of servings.
